SEQUENCE LISTING (11 sequences)

SEQ ID NO: 1 - DNA, 750 bp, Citrus sinensis
atggatattgcacatttcttgttcggtgtttttggaaatgctactgctttatttctcttcttggctccaacaatcacatttaggaggattgtaagaaggaaatccacagagcaattctctggaattccttatgtgatgaccttgcttaattgtctcctctctgcctggtatggtctcccttttgtgtcgaagaacaacattttggtatcaacaatcaatggaactggttcagcaattgagatcatatatgttttgatcttcctactctttgcgccaaagaaggagaaagcgaaaatctttgggcttttcatgcttgttctcacagtcttcgctgctgttgccttggtgtctctcttagcctttcatggcaacgctagaaagattttctgtggctttgctgctaccattttctctattatcatgtatgcctcacctctttctatcatgagaatggtgattaaaacaaagagtgttgagttcatgccatttttcttgtcactatttgtcttcctgtgtggcacctcttggttcgtgtttggccttcttggccgtgacccttttgtggccgttcccaatggatttgggtgtgggctaggcacaatgcagttgattttgtacttcatctatcacaagaagggtgagcctgagaaaccatcggcagcaaatgggtctgttgaaatgggccaagagaaaccattagaaggaaccaagatggctaacggcaacggggctctagttgaacaagtttga

SEQ ID NO: 2 - DNA, 1111 bp, Citrus sinensis
gtgcgctcatcttcctagaaggtgcaaatacttttgctctcttaaattttttatcactatgctatctgcatatatagataattaataaaatcaaattatggataccaaagttctgtcctcgggaatccggtacacaaacttgccggagggctacgtaaggccagaatcggagagaccaaatctgtctgaagtttctgaatgcaaaaacgttcctgttattgatttggcttgtgatgatagaagcttaattgtgcaacaagttgctgatgcttgcaagaattatggatttttccaggcgataaatcacgaagtgcctttagaaacagtggaaagagtcttagaagtggcgaaagagttttttaacctgccagtcgaggagaagctgaagctctactcagatgatccatcaaagacaatgagattgtcaacgagttttaatgtcaacaaggagaaagttcacaattggagagattacctcagacttcactgctatcctcttgacaagtatgtgcctgagtggccttcaaatccatctaccttcaaggaatttgtaagtacttattgttcagaagttcgaggactcggctatagagtgctggaattaatatcagagagcttaggtttggaaaaagattacataaagaaagtgttgggtgaacaaggacagcatatggctgtgaacttttacccaccatgtccagagccagagcttacttatggattgccaggacacactgacccaaatgcccttaccattttattgcaagatttagaagtggcaggtcttcaagttctcaaagacgacaaatgggttgctgttaatccccttcctaatgcctttgtcattaatattggtgatcaactgcaggcattgagtaatggaaggtataagagtgtgtggcatagagctattgtgaatgctgaaaaggcaaggatgtcggttgcttcattcctttgcccaaataatgatgcaatgatcagccctccaaaagcattaacagaggatggatctggagccgtatacagagatttcacatatgctgaatattacagcaagttttggagcagaaacttggaccaagaacattgtttggaacttttcaagaactag

SEQ ID NO: 3 - DNA, 20 bp, Artificial Sequence
  Primer
agcgaaaatctttgggcttt

SEQ ID NO: 4 - DNA, 22 bp, Artificial Sequence
  primer
gaaagaggtgaggcatacatga

SEQ ID NO: 5 - DNA, 22 bp, Artificial Sequence
  primer
tcatcttcctagaaggtgcaaa

SEQ ID NO: 6 - DNA, 20 bp, Artificial Sequence
  primer
cctcgactggcaggttaaaa

SEQ ID NO: 7 - DNA, 153 bp, Citrus sinensis
agcgaaaatctttgggcttttcatgcttgttctcacagtcttcgctgctgttgccttggtgtctctcttagcctttcatggcaacgctagaaagattttctgtggctttgctgctaccattttctctattatcatgtatgcctcacctctttc

SEQ ID NO: 8 - DNA, 469 bp, Citrus sinensis
tcatcttcctagaaggtgcaaatacttttgcgctctttaattttttatttatcactatggtatctgcatatatagataattaataaaatcaaattatggataccaaagttctgtcctcgggaatccggtacacaaacttgccggagggctacgtaaggccagaatcggagagaccaaatctgtctgaagtttctgaatgcgaaaacgttcctgttattgatttggcttgtgatgatagaagcttaattgtgcaacaagttgctgatgcttgcaagaattatggatttttccaggtttgtgttcaaaatttccttgccctaaatcatttgagtttgttatatagatgtataaacgcgttaatcagtcatatattggttgcatatacaggcgataaatcacgaagtgcctttagaaacagtggaaagagtcttagaagtggcgaaagagttttttaacctgccagtcgagg

SEQ ID NO: 9 - DNA, 23 bp, Artificial Sequence
  Guide Sequence
ccttggtgtctctcttagccttt

SEQ ID NO: 10 - DNA, 23 bp, Artificial Sequence
  Guide Sequence
cctcgggaatccggtacacaaac

SEQ ID NO: 11 - DNA, 23 bp, Artificial Sequence
  Guide Sequence
agtggaaagagtcttagaagtgg

(19) United States
(12) Patent Application Publication
(10) Pub. No.: US 2021/0189410 A1
(43) Pub. Date: Jun. 24, 2021

(54) TARGETED EDITING OF CITRUS GENES FOR DISEASE RESISTANCE

(71) Applicant: UNIVERSITY OF FLORIDA RESEARCH FOUNDATION, INCORPORATED, Gainesville, FL (US)

(72) Inventors: Zhanao DENG, Wimauma, FL (US); Saroj PARAJULI, Wimauma, FL (US)

(21) Appl. No.: 17/105,928

(22) Filed: Nov. 27, 2020

Related U.S. Application Data
(60) Provisional application No. 62/941,351, filed on Nov. 27, 2019.

(51) Int. Cl. C12N 15/82

(52) U.S. Cl. CPC C12N 15/8213; C12N 15/8281

(57) ABSTRACT

Disclosed herein our novel methods of increasing resistance of Citrus plants to diseases, in particular, citrus canker disease caused by Xanthomonas citri ssp. citri and Huanglongbing disease. Some embodiments relate to novel methods of altering gene sequence, structure and expression of certain disease susceptibility genes in the Citrus plant. Other embodiments relate to gene constructs equipped to be introduced into Citrus cells and direct modifications to target gene sequences.

### GOVERNMENT SUPPORT

This invention was made with government support under 2015-70016-23027 & 2017-70016-26051 awarded by The United States Department of Agriculture, National Institute of Food & Agriculture. The government has certain rights in the invention.

### BACKGROUND

Citrus (Citrus L.) is one of the most important fruit crops in the United States (U.S.) and in the world. Sweet oranges, grapefruit, pummelos (pomelos), lemons, limes, mandarins, tangerines, etc. are different species of citrus. Citrus production in the U.S. and in the world is facing significant challenges from multiple diseases. Among them are citrus cancer, Huanglongbing (HLB, or citrus greening) and citrus variegated chlorosis (CVC). Citrus canker is a serious bacterial disease worldwide. It is caused by the bacterial pathogen Xanthomonas citri ssp. citri (Xcc). Infection of this pathogen can cause severe defoliation, shoot dieback, and fruit drop in citrus, resulting in severe losses of crop yield (Ference et al., 2018; Gochez et al., 2020). Infected citrus fruit are of lower commercial value and can become unmarketable. The causal agent of HLB is presumed to be phloem-limited bacteria of Candidatus Liberibacter asiaticus (CLas). Both diseases have caused remarkable losses in the citrus industries in the world. For example, citrus canker was re-introduced to Florida in 1995, and $1.3 billion was spent on eradication effort, which had to be abandoned in 2006 after hurricanes spread the pathogen throughout the state (Gottwald et al., 2002; Graham et al., 2004). HLB was first found in Florida in 2005. Since then, orange acreage and yield in Florida have decreased by 26% and 42%. In Florida alone, HLB has cost the citrus industry $4.5 billion in lost economic activity and 8257 jobs (National Research Council, 2010). The cost of growing citrus in Florida has tripled the cost prior to HLB. The Florida citrus industry has spent more than $117 million over recent years on research for solution to citrus greening.

The majority of citrus cultivars are susceptible to citrus canker. Essentially all the citrus cultivars are susceptible to HLB. Enhancing citrus resistance to diseases citrus canker and HLB has been high priority to the citrus industry in Florida, other citrus-producing states in the U.S., and other major citrus-producing countries in the world.

### BRIEF DESCRIPTION OF DRAWINGS

FIG. 1A. Schematic map of the CRISPR/Cas9 plasmid pS1g1g2-Cas9 (within the left and right borders only) assembled for editing the SWEET1 gene in Citrus. The GFP-NPTII fusion protein gene is driven under the 2X Casmv 35S promoter, the SpCas9 gene is driven under the Camv 35S promoter, and the gRNA (SWT1_gRNA1) targeting the SWEET1 gene is driven under the At U6 promoter. Abbreviations: GFP-NPTII: green fluorescence protein and neomycin phosphotransferase fusion protein gene; Casmv: cassava mosaic virus; Camv: cauliflower mosaic virus; At: Arabidopsis thaliana; HSP: heat shock protein gene; U6: ubiquitin6; tRNA: transfer RNA; and SWT1_gRNA1: guide RNA1 targeting the SWEET1 gene.

FIG. 1B. Schematic map of the pD6g1g2-HyCas9 CRISPR/Cas9 plasmid (within the left and right borders only) assembled for editing the DMR6 gene in Citrus. The GFP-NPTII fusion protein gene is driven under the 2x Casmv 35S promoter, the Hypa-Cas9 gene is driven under the Camv 35S promoter, and the two gRNAs (dmr6_gRNA1 and dmr6_gRNA2) targeting the DMR6 gene are driven under the At U6 promoter. Abbreviations: GFP-NPTII: green fluorescence protein and neomycin phosphotransferase fusion protein gene; Casmv: cassava mosaic virus; Camv: cauliflower mosaic virus; At: Arabidopsis thaliana; HSP: heat shock protein gene; U6: ubiquitin6; tRNA: transfer RNA; dmr6_gRNA1 and dmr6_gRNA2: guide RNA1 and guide RNA2 targeting the DMR6 gene; and Hypa: hyper-accurate.

FIGS. 1C-1F. Agrobacterium-mediated transformation and regeneration of SWEET1-edited transgenic ‘Duncan’ grapefruit plants. FIG. 1C) Agrobacterium-infected epicotyl stem segments; FIG. 1D) GFP expression in transgenic shoots; FIG. 1E) micro-grafting of GFP-positive shoot onto ‘Carrizo’ citrange rootstock to produce complete plantlets, and FIG. 1F) transgenic SWEET1 mutant plant established in a container filled with potting mixture.

FIG. 2A. Mutations and mutation frequencies in five different SWEET1-edited ‘Duncan’ grapefruit mutant lines in the SWT1-gRNA1-targeted region of the SWEET1 gene. WT: wildtype non-transformed ‘Duncan’. Orange-colored letters represents gRNA; blue-colored three nucleotides “CCT” are the protospacer adjacent motif (PAM); black-colored nucleotide “T” represents insertion of base “T”; and each hyphen represents deletion of a single nucleotide.

FIG. 2B. Mutations and mutation frequencies in three different DMR6-edited ‘Duncan’ grapefruit mutant lines in the dmr6-gRNA1-targeted region of the DMR6 gene. WT: wildtype non-transformed ‘Duncan’ grapefruit. Orange-colored italic letters represents the whole or part of the gRNA; the three blue-colored nucleotides “CCT” make up the protospacer adjacent motif (PAM); black-colored nucleotide in the gRNA region represents insertion of a base; and each hyphen represents deletion of a single nucleotide.

FIG. 2C. Mutations and mutation frequencies in six different DMR6-edited ‘Carrizo’ citrange mutant lines in the dmr6-gRNA1-targeted region of the DMR6 gene. WT: wild-type non-transformed ‘Carrizo’ citrange. Orange-colored italic letters represents dmr6-gRNA1; blue-colored three nucleotides “CCT” are the protospacer adjacent motif (PAM); black-colored nucleotides in the gRNA region represents insertion of bases; and each hyphen represents deletion of a single nucleotide.

FIG. 3A. Citrus canker lesions on leaves of wildtype (WT) ‘Duncan’ grapefruit and five SWEET1-edited lines (DS1, DS2, DS4, DS5 and DS7) at 4, 8 and 12 days after inoculation (DAI) with Xanthomonas citri ssp. citri (Xcc). Each leaf was inoculated at two sites across the mid vein by infiltrating an Xcc cell suspension (10⁸ CFU/mL). WT began to show two large lesions on each leaf 4 days after inoculation. Lines DS1, DS2, DS5 and DS7 showed 60.0% to 90.7% reduction in lesion areas at 12 DAI, compared to the WT. DAI: days after inoculation; WT: wildtype non-edited ‘Duncan’ grapefruit.

FIG. 3B. Citrus canker lesions on leaves of the wildtype (WT) and three DMR6-edited ‘Duncan’ grapefruit mutant lines (DD9, DD15 and DD19) at 4, 8 and 12 days after inoculation (DAI) with Xanthomonas citri ssp. citri (Xcc). Each leaf was inoculated at two sites across the mid vein by infiltrating an Xcc cell suspension (10⁸ CFU/mL). WT began to show two large lesions on each leaf 4 days after inoculation. All mutant lines showed much smaller lesions. Lines DD9 and DD19 showed 71.2% and 98.2% reduction in lesion area, respectively, compared to the WT. DAI: days after inoculation; WT: wildtype non-edited ‘Duncan’ grapefruit.

FIG. 3C. Citrus canker lesions on leaves of the wildtype (WT) and six DMR6-edited ‘Carrizo’ citrange mutant lines (D3, D4, D7, D8, D10 and D12) at 10, 15 and 20 days after inoculation (DAI) with Xanthomonas citri ssp. citri (Xcc). Each leaf was inoculated at two sites across the mid vein by infiltrating an Xcc cell suspension (10⁸ CFU/mL). WT began to show two lesions on each leaf 10 days after inoculation. Mutant lines D4, D7, D10 and D12 showed 91.2% to 98.6% reduction in lesion area at 20 DAI, compared to the wildtype non-transformed, non-edited ‘Carrizo’ citrange. DAI: days after inoculation; WT: wildtype non-edited ‘Carrizo’ citrange.

FIG. 4A. Mean citrus canker lesion area (mm² per leaf inoculated at two sites) on leaves of wildtype (WT) ‘Duncan’ grapefruit and five SWEET/-edited mutant lines (DS1, DS2, DS4, DS5 and DS7) at 4, 8 and 12 days after inoculation (DAI) with Xanthomonas citri ssp. citri (Xcc). Mutant lines DS1, DS2, DS5 and DS7 showed much smaller lesions and 60.0% to 90.7% reduction in lesion areas at 12 DAI, compared to WT. DAI: days after inoculation; WT: wildtype non-edited ‘Duncan’ grapefruit.

FIG. 4B. Mean citrus canker lesion area (mm² per leaf with two inoculated sites) on leaves of wildtype (WT) ‘Duncan’ grapefruit and three DMR6-edited mutant lines (DD9, DD15 and DD19) at 4, 8 and 12 days after inoculation (DAI) with Xanthomonas citri ssp. citri (Xcc). All three mutant lines showed much smaller lesions; lines DD9 and DD19 showed 71.2% and 98.2% reduction in lesion size at 12 DAI, compared to the wildtype. DAI: days after inoculation; WT: wildtype non-edited ‘Duncan’ grapefruit control.

FIG. 4C. Mean citrus canker lesion area (mm² per leaf with two inoculated site) on leaves of wildtype (WT) ‘Carrizo’ citrange and six DMR6-edited mutant lines (D3, D4, D7, D8, D10 and D12) at 10, 15 and 20 days after inoculation (DAI) with Xanthomonas citri ssp. citri (Xcc). All mutant lines showed much smaller lesions; mutant lines D4, D7, D10 and D12 showed 91.2% to 98.6% reduction in lesion area at 20 DAI, compared to the WT. DAI: days after inoculation; WT: wildtype non-edited ‘Carrizo’ citrange.

FIG. 5A. Bacterial counts (log10 of colony-forming units per cm²) in leaves of wildtype (WT) ‘Duncan’ grapefruit and five SWEET/-edited mutant lines (DS1, DS2, DS4, DS5 and DS7) 8, 12, or 16 days after inoculation with Xanthomonas citri ssp. citri (Xcc). Lines DS1, DS2, and DS7 showed significantly lower bacterial counts; DS2 and DS7 showed the lowest Xcc bacterial counts, with greater than 99.9% reduction (or 3.97-4.67 Log10 unit reduction) in Xcc cell counts at 16 DAI, compared to WT. DAI: Days after inoculation.

FIG. 5B. Bacterial counts [Log10 of colony-forming units (CFU) per cm²] in leaves of wildtype (WT) ‘Duncan’ grapefruit and three DMR6-edited mutant lines (DD9, DD15 and DD19) 10 and 20 days after inoculation with Xanthomonas citri ssp. citri (Xcc). Lines DD9 and DD19 showed the lowest Xcc bacterial counts, with greater than 99% reduction (or 4.04 and 3.36 Log10 unit reduction) in Xcc cell counts (CFU) at 20 DAI, compared to WT. DAI: Days after inoculation.

FIG. 5C. Bacterial counts (Log10 of colony-forming units per cm²) in leaves of wildtype (WT) ‘Carrizo’ citrange and six DMR6-edited mutant lines (D3, D4, D7, D8, D10 and D12) at 10 and 20 days after inoculation with Xanthomonas citri ssp. citri (Xcc). All mutant lines showed lower bacterial cell counts; lines D7, D10 and D12 showed significantly lower Xcc bacterial cell counts, with greater than 99% reduction (or 3.72-4.92 Log10 unit reduction) in Xcc cell counts (CFU) at 20 DAI, compared to WT. DAI: Days after inoculation.

FIG. 6. Salicylic acid content (ng/mg) in fresh leaves of wildtype (WT) ‘Duncan’ grapefruit, two SWEET/-edited mutant lines (DS2 and DS7) and two DMR6-edited mutant lines (DD9 and DD19) before (BI) and 24 hours after inoculation (HAI) with Xanthomonas citri ssp. citri (Xcc). BI: before inoculation; HAI: hours after inoculation; SA: salicylic acid; WT: wild-type; and FW: fresh weight.

FIG. 7. Relative expression level of the non-expressor of pathogenesis-related 1 (NPR1) gene in the leaves of wildtype ‘Carrizo’ citrange (WT) and two DMR6-edited ‘Carrizo’ mutant lines (D10 and D12) before inoculation (0 DAI) and 10, 15 and 20 days after inoculation (DAI) with Xanthomonas citri ssp. citri. Both mutant lines D10 and D12 showed much increased expression of the NPR1 gene at 10 days after inoculation (DAI), compared to the WT. The glyceraldehyde 3-phosphate dehydrogenase (GAPDH) gene was used as a reference gene to calculate the relative gene expression level. WE: wild-type ‘Carrizo’.

### DETAILED DESCRIPTION

### Overview

Disclosed herein is a strategy is to identify genes involved in disease susceptibility, edit them to alter their DNA sequence and gene structure and knock down or knock out the expression and function of their normal peptide or protein products, so that the gene-edited citrus plants will become resistant to diseases. As disclosed herein, two genes in citrus have been identified for targeting: SWEET1 and DMR6. In rice, SWEET (Sugars will eventually be exported transporters) genes are involved in rice susceptibility to Xanthomonas oryzae pv. oryzae (Xoo). Knocking-out SWEET11, SWEET13 and SWEET14 genes increased rice resistance in Xoo (Xu et al., 2019). DMR6 gene encodes a salicylate 3-hydroxylase (Zhang et al., 2017). Its protein is a repressor of plant immunity (Zeilmaker et al., 2015). It regulates negatively the expression of plant defense genes such as PR-1, PR-2, and PR-5. It is required for susceptibility to a number of pathogens, including susceptibility to the downy mildew pathogen Hyaloperonospora in Arabidopsis, susceptibility to Pseudomonas syringae pv. Tomato DC3000 (Zhang et al., 2017), and susceptibility to the oomycete Phytophthora capsici (Van Damme et al., 2005). Disruptive mutations of DMR6 resulted in resistance to diseases (Zeilmaker et al., 2015). CRISPR-Cas9 mediated mutagenesis of a DMR6 ortholog in tomato resulted in broad-spectrum resistance to P. syringae, P. capsica, and Xanthomonas gardneri and X. perforans (del Toledo Thomazella et al., 2016). It has been determined through gene expression studies that SWEET genes may be potentially involved in citrus susceptibility to citrus canker and that DMR6 is involved in citrus susceptibility to HLB (Wang et al., 2016; Ibanez et al., 2020).

To determine the roles of citrus SWEET and DMR6 genes in citrus canker and HLB susceptibility, SWEET1 and DMR6 were targeted for CRISPR/Cas9-mediated mutagenesis and knocking-down or knocking-out in two citrus genotypes, ‘Duncan’ grapefruit (Citrus x paradisi) and ‘Carrizo’ citrange (hybrid of Citrus sinensis and Poncirus trifoliata). ‘Duncan’ grapefruit and ‘Carrizo’ citrange mutants resulting from CRISPR editing of SWEET1 and DMR6 have been assessed for resistance to citrus canker.

Five ‘Duncan’ grapefruit mutants carrying mutations in the SWEET1, three ‘Duncan’ grapefruit mutants carrying mutations in the DMR6 gene, and six ‘Carrizo’ citrange mutants carrying mutations in the DMR6 gene were tested. At least two SWEET1 mutants (‘Duncan’ grapefruit line DS2 and DS7) and five DMR6 mutants (‘Duncan’ grapefruit line DD9 and DD19, and ‘Carrizo’ citrange line D7, D10 and D12) have shown high levels of resistance to a Florida Xcc strain and the citrus canker it caused.

Compared to wildtype ‘Duncan’ grapefruit, the citrus canker lesion (raised, discolored, necrotic) area on leaves of the SWEET1 mutants DS2 and DS7 was reduced by 73.0% and 90.7% at 12 days after Xcc inoculation (DAI). Bacterial cell counts or populations in leaves of the SWEET1 mutants DS2 and DS7 was reduced by greater than 99.90% (3.97-4.67 Log10 units) at 16 DAI with Xanthomonas citri ssp. citri, compared to wildtype ‘Duncan’ grapefruit. The primary type of mutation induced in ‘Duncan’ grapefruit line DS2 and DS7 is a one-base deletion (of T) (88.26% and 94.73%, respectively). Minor mutations include two-base deletion and one-base insertion. The total mutation frequency in the leaf cells of DS2 and DS7 was 100%. Compared to wildtype ‘Duncan’ grapefruit, SWEET1 mutant lines DS2 and DS7 exhibited 12% to 15% higher salicylic acid (SA) contents before inoculation with Xcc and approximately 25% to 35% higher SA contents 24 hours after Xcc inoculation.

Compared to the wildtype ‘Duncan’ grapefruit, citrus canker lesion area on the leaves of the DMR6 mutants DD9 and DD19 was reduced by 71.2% and 98.2% at 12 days after inoculation with Xanthomonas citri ssp. citri. Bacterial cell counts or populations in leaves of the DMR6 mutants DD9 and DD19 was reduced by greater than 99.9% (3.36-4.04 Log10 units) at 20 days after inoculation with Xanthomonas citri ssp. citri, compared to wildtype ‘Duncan’ grapefruit. Three types of mutations induced in ‘Duncan’ grapefruit line DD9 were four-base deletion (deletion of GAAT) (15.33%), ten-base or larger deletion (10.96%), and one-base insertion (12.17%). The total mutation frequency in the leaf cells of DD9 was 26.29% to 38.46%. Four types of mutation induced in ‘Duncan’ grapefruit line DD19 are one-base deletion (of G) (14.27%), ten-base or larger deletion (42.28%), one-base insertion (of G) (15.23%), and five-base deletion (2.39%). The total mutation frequency in the leaf cells of mutant line DD19 was 71.78% to 74.17%. Compared to wildtype ‘Duncan’ grapefruit, DMR6 mutant lines DD9 and DD19 exhibited significantly higher (95% to 116% higher) salicylic acid (SA) contents before inoculation with Xcc and approximately 30% higher SA contents 24 hours after Xcc inoculation.

Compared to the wildtype ‘Carrizo’ citrange, citrus canker lesion area on the leaves of the DMR6 mutants D4, D7, D10 and D12 was reduced by 90% or greater at 20 days after inoculation with Xanthomonas citri ssp. citri. Bacterial cell counts or populations in leaves of the DMR6 mutants D7, D10 and D12 was reduced by 99% or greater (2.81-4.92 Log10 units) at 20 days after inoculation with Xanthomonas citri ssp. citri, compared to wildtype ‘Duncan’ grapefruit. The primary types of mutations induced in ‘Carrizo’ line D4 were one-base insertion (of G) (51.34%) and two-base deletion (of GA) (33.06%). Minor mutations included one-base deletion (of A) (2.16%) and five-base deletion (2.47%). The total mutation frequency in the leaf cells of mutant line D4 was 86.54% to 89.01%. The primary types of mutations induced in ‘Carrizo’ mutant line D7 was one-base deletion (of G) (49.74%) and one-base insertion (of A) (31.31%). The minor mutation in line D7 was five-base deletion (3.52%). The total mutation frequency in the leaf cells of line D7 was 81.06% to 84.58%. The primary type of mutation induced in ‘Carrizo’ line D10 was ten-base or larger deletion (85.37%), and the minor mutation was two-base insertion (of GA) (6.40%). The total mutation frequency in the leaf cells of line D10 was 91.77%. The primary types of mutations induced in ‘Carrizo’ line D12 were four-base deletion (of GAAT) (70.70%) and seven-base deletion (71.11%); the minor mutation induced in line D12 was one-base insertion (of T) (28.15%). The total mutation frequency in the leaf cells of ‘Carrizo’ mutant line D12 was 98.85% to 100%. Compared to wildtype ‘Carrizo’ plants, DMR6 mutant lines D10 and D12 expressed 16-to 17-fold higher levels of the non-expressor of pathogenesis-related 1 (NPR1) gene 10 days after inoculation with Xcc and 20% to 45% higher levels of NPR1 15 days after Xcc inoculation.

While the invention is susceptible to various modifications and alternative forms, specific embodiment thereof has been shown by way of example in the drawings and will be described in detail below. It should be understood, however that it is not intended to limit the invention to the particular forms disclosed, but on the contrary, the invention is to cover all modifications, equivalents, and alternatives falling within the spirit and the scope of the invention. All documents, patents, journal articles and other materials cited herein are incorporated in their entirety to the extent not inconsistent with the teachings herein.

### Definitions

Practice of the methods, as well as preparation and use of the compositions disclosed herein employ, unless otherwise indicated, conventional techniques in molecular biology, biochemistry, DNA and RNA structure and analysis, computational chemistry, cell culture, recombinant DNA and related fields as are within the skill of the art. These techniques are fully explained in the literature. See, e.g., Sambrook et al. MOLECULAR CLONING: A LABORATORY MANUAL, 2d ed., Cold Spring Harbor Laboratory Press, 1989; 3d ed., 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego.

The term “citrus” refers to any known variety, cultivar, breeding line or accession of plants in the genus Citrus and sexually compatible genera Fortunella, Poncirus, Microcitrus and Eremocitrus. In some recent classification, plants of Fortunella, Poncirus, Microcitrus and Eremocitrus have been placed in the genus Citrus. Citrus varieties contemplated by this disclosure include, but are not limited to, cultivated citrus types such as sweet oranges, blood oranges, navel oranges, round oranges, grapefruit, pomelos or pumellos, citrons, lemons, limes, mandarins, Clementine mandarins, Satsuma mandarins, tangerines, tangors, tangelos, trifoliate oranges, citranges, citrumelos, or the like.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

The term “alter(ing) DNA sequence, gene structure and function” refers to 1) introducing a change (deletion or insertion, i.e. indel) of deoxynucleotide(s) in the gene's coding sequence (CDS), 2) introducing a change (indel) of deoxynculeotide(s) in the promoter region, and 3) introducing a change (indel) of deoxynculeotide(s) in the gene's 5′ untranslated region (UTR). Such changes in the gene's coding sequence is expected to result in 1) a change (deletion or insertion) of amino acid(s) in the gene product encoded by the gene, and/or 2) a frameshift of the open reading frame of the gene leading to a pre-matured polypeptide, non-functional polypeptide or a function-impacted polypeptide. Such changes in the gene's promoter region and/or in the gene's 5′ UTR is expected to result in 1) a change of the expression pattern or level of the gene, 2) a loss of the gene's promoter region to bind to pathogen's effector or virulence factor protein(s), and/or 3) a loss of the gene's responsiveness to pathogen's infection and interaction(s).

The term “reducing expression of a gene” refers to a decrease or elimination of expression of a gene product encoded by a gene. In a specific example, reducing expression of a gene can be achieved by knocking down or knocking out the gene out of its normal structure and function.

The term “sequence” refers to a nucleotide sequence of any length, which can be DNA or RNA; can be linear, circular or branched and can be either single-stranded or double stranded.

The term a “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In certain embodiments, the degree of homology between the two sequences is sufficient to allow homologous recombination between the two sequences. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively.

Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects sequence identity. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenB ank+EMBL+DDBJ+PDB+GenB ank CDS translations+Swiss protein+Spupdate+PIR. Details of these programs can be found at the following internet address: http://www.ncbi.nlm.gov/cgi-bin/BLAST. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Conditions for hybridization are well-known to those of skill in the art. Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

### Description of Specific Embodiments

In one embodiment, provided is a method of altering the DNA sequence, changing the gene structure and product, and/or reducing the expression of at least one gene product of a gene that involves introducing into a citrus plant cell an engineered, non-naturally occurring gene editing system comprising one or more vectors. The citrus plant cell contains an expressible DNA molecule that encodes the gene and includes a target gene sequence. The gene editing system introduced includes: (a) a first regulatory element operable in a plant cell operably linked to a nucleotide sequence encoding a Type-II CRISPR-associated nuclease, and (b) a second regulatory element operable in a plant cell operably linked to at least one nucleotide sequence encoding a CRISPR-Cas system guide RNA (gRNA) that hybridizes (binds or has at least 90% nucleotide identity) with the target sequence. The gene editing system may comprise one or more vectors, and in a specific embodiment, components (a) and (b) are located on the same or different vectors of the system. The CRISPR-associated nuclease cleaves the citrus DNA molecule such that the NA sequence and structure of the gene is altered in its coding region, promoter and/or 5′ untranslated region, and/or the expression level and/or pattern of the gene is altered. Specific genes targeted for DNA sequence alteration and reduced expression includes SWEET1 and/or DMR6 of Citrus. In a specific embodiment, the coding sequence of the DNA Molecule is encoded by SEQ ID NO: 1 or SEQ ID NO: 2.

In a specific embodiment, the sequence encoding a Type-II CRISPR-associated nuclease is operably linked to a terminator sequence functional in a citrus plant cell. In a specific embodiment, the type-II CRISPR-associated nuclease is Cas9.

The second regulatory element may comprise a DNA-dependent RNA polymerase III (Pol III) promoter sequence. In a specific example, the Pol III promoter sequence pertains to an Arabidopsis U6 promoter nucleotide sequence.

In a specific embodiment, the gRNA is CCTTGGTGTCTCTCTTAGCCTTT. In another embodiment, the gRNA is CCTCGGGAATCCGGTACACAAAC or AGTGGAAAGAGTCTTAGAAGTGG, or a combination.

According to further embodiment, disclosed another method of altering the DNA sequence, changing the gene product, and/or reducing expression of at least one gene product of a gene that involves introducing into a citrus plant cell a CRISPR-Cas-ribonucleoprotein complex (CRISPR-Cas-RNP). The citrus plant cell contains and expresses a DNA molecule encoding the gene and includes a target sequence. The CRISPR-Cas-RNP includes a CRISPR-Cas system guide RNA (gRNA) that hybridizes (binds or has at least 90% nucleotide identity) with the target sequence, and a class-II CRISPR-associated nuclease. The gene targeted is SWEET1 or DMR6, or both. In a specific embodiment, introducing comprises PEG-transfection, wherein the w/v percent of PEG is between 30-50%, or about 40%. The class II CRISPR-associated nuclease may pertain to cfp1. Specific examples of cfp1 include FnCpf1 from Francisella novicida, AsCpf1 from Acidaminococcus sp, and LbCpf1 from Lachnospiraceae bacterium.

Also disclosed are citrus plant cells with altered DNA sequence, changed gene structure, and/or reduced expression of a gene product as produced by the methods described above. Further, embodiments include plants, plant tissues, or seeds comprising the plant cells having reduced expression.

A further embodiment pertains to a plasmid comprising: (a) a first regulatory element operable in a plant cell operably linked to a nucleotide sequence encoding a Type-II CRISPR-associated nuclease, and (b) a second regulatory element operable in a plant cell operably linked to at least one nucleotide sequence encoding a CRISPR-Cas system guide RNA (gRNA) that hybridizes (binds or has at least 90% nucleotide identity) with a target sequence of a gene, wherein the guide RNA hybridizes with the target sequence and the CRISPR-associated nuclease cleaves the target sequence, whereby the DNA sequence, gene structure, expression of the gene is altered, and wherein the gene is SWEET1 or DMR6. The sequence encoding a Type-II CRISPR-associated nuclease can be operably linked to a terminator sequence functional in a plant cell. In a specific embodiment, the type II CRISPR-associated nuclease is Cas9. In another specific embodiment, the second regulatory element comprises a DNA-dependent RNA polymerase III (Pol III) promoter sequence.

The citrus plant cell wherein a DNA molecule has been altered to change the gene structure and reduce the gene expression includes, but is not limited to, a cell of a sweet orange, bitter orange, blood orange, naval orange, grapefruit, pomelo, citron, lemon, lime, Clementine, mandarin, tangerine, tangelo, tangor, trifoliate orange, trifoliate orange hybrid, and kumquat plant cell. In a specific embodiment, the citrus plant cell is ‘Duncan’ grapefruit.

A further embodiment pertains to a citrus plant cell or citrus plant comprising a cell, wherein the cell has been modified to include a mutation in the SWEET1 and/or DMR6 gene, such that the DNA sequence and the structure of the gene are changed, and/or the expression of the gene is reduced.

In yet a further embodiment, provided is a method of increasing resistance against citrus canker disease in a citrus plant comprising altering the DNA sequence and the structure and/or reducing expression of the SWEET1 gene, or the DMR6 gene, or both in cells of the citrus plant. In a specific embodiment, reducing expression comprises imparting a mutation in the SWEET1 gene or DMR6 gene. In one specific example, imparting a mutation comprises introducing into one or more cells of the citrus plant a gene editing system comprising one or more vectors, comprising: (a) a first regulatory element operable in a plant cell operably linked to a nucleotide sequence encoding a Type-II CRISPR-associated nuclease, and (b) a second regulatory element operable in a plant cell operably linked to at least one nucleotide sequence encoding a CRISPR-Cas system guide RNA (gRNA) that hybridizes (binds or has at least 90% nucleotide identity) with a portion of the SWEET1 gene or DMR6 gene. In a second example, imparting a mutation comprises introducing into one or more cells of the citrus plant a CRISPR-Cas-ribonucleoprotein complex (CRISPR-Cas-RNP) comprising a CRISPR-Cas system guide RNA (gRNA) that hybridizes with a portion of the SWEET1 gene or DMR6 gene, and a class-II CRISPR-associated nuclease.

### Genetic Transformation Methods and Plant Regeneration

Gene transfer and genetic transformation methods for introducing engineered gRNA-Cas9 constructs into plant cells include, but are not limited to, protoplast transformation through calcium-, polyethylene glycol (PEG)- or electroporation-mediated uptake of naked DNA (see Paszkowski et al. (1984) EMBO J3:2717-2722; Potrykus et al. (1985) Molec. Gen. Genet. 199:169-177; Fromm et al. (1985) Proc. Nat. Acad. Sci. USA 82:5824-5828; and Shimamoto (1989) Nature 338:274-276) and electroporation of plant tissues (D'Halluin et al. (1992) Plant Cell 4:1495-1505). Additional methods for plant cell transformation include microinjection, silicon carbide mediated DNA uptake (Kaeppler et al. (1990) Plant Cell Reporter 9:415-418), and microprojectile bombardment (see Klein et al. (1988) Proc. Nat. Acad. Sci. USA 85:4305-4309; and Gordon-Kamm et al. (1990) Plant Cell 2:603-618). According to certain embodiments, gene constructs carrying gRNA-Cas9 nuclease can be introduced into plant cells by various methods, which include but are not limited to PEG- or electroporation-mediated protoplast transformation, tissue culture or plant tissue transformation by biolistic bombardment, or the Agrobacterium-mediated transient and stable transformation.

Target gene sequences for genome editing and genetic modification can be selected using methods known in the art, and as described elsewhere in this application. In a preferred embodiment, target sequences are identified that include or are proximal to protospacer adjacent motif (PAM). Once identified, the specific sequence can be targeted by synthesizing a pair of target-specific DNA oligonucleotides with appropriate cloning linkers, and phosphorylating, annealing, and ligating the oligonucleotides into a digested plasmid vector, as described herein. The plasmid vector comprising the target-specific oligonucleotides can then be used for transformation of a plant. In specific embodiments, the target gene sequences comprise a disease susceptibility gene.

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker or reporter marker genes (such as GFP or GUS gene) which has been introduced together with the desired nucleotide sequences. Plant regeneration from cultured protoplasts is described in Evans, et al., “Protoplasts Isolation and Culture” in Handbook of Plant Cell Culture, pp. 124-176, Macmillian Publishing Company, New York, 1983; and Binding, Regeneration of Plants, Plant Protoplasts, pp. 21-73, CRC Press, Boca Raton, 1985. Regeneration can also be obtained from plant callus, explants, organs, pollens, embryos or parts thereof. Such regeneration techniques are described generally in Klee et al (1987) Ann. Rev. of Plant Phys. 38:467-486.

### Non-Transgenic Plant Cell Transfection of Ribonucleoprotein (RNPs) Complexes

The methods for non-transgenic plant cell transfection do not depend on a particular method for introducing RNP into the cell. The RNP is provided to the cells and taken up into the cell interior. Introduction of the RNP may be accomplished by any method known, which permits the successful introduction of the RNP into the cells. Methods include but are not limited to such methods as transfection, microinjection, electroporation, nucleofection and lipofection. Preferably, a PEG transfection is used, as further detailed herein below.

CRISPR-Cas RNPs can be produced fast and delivered directly to the cells as completely functional complexes. Indeed, they are instantaneously active after transfection, and rapidly breakdown inside the cell. This rapid breakdown kinetics permits CRISPR-Cas RNPs to modify the target genes with lower off-target effects. However a successful editing using CRISPR-Cas RNPs always rely on an efficient CRISPR RNA (crRNA) and a solid delivery method which can avoid the influence of cell wall as a major barrier. In this regard, plant protoplasts generated by the removal of cell wall using enzymatic digestion provides a promising strategy to improve the efficiency of CRISPR-Cas RNP systems, since the lack of the cell wall makes it possible to employ transfection or electroporation for RNPs and/or nucleic acid deliveries. Moreover, the protoplast can be used to analyze target site mutagenesis efficiency and can be regenerated into plant.

Among the CRISPR systems, Cpf1 as the effector of the CRISPR locus is identified as a class two CRISPR which recognizes the target DNA region via protospacer adjacent motif (PAM) scanning (PAM in Cpf1 is highly specific to the 5′-TTTV-3′). CRISPR-Cpf1 systems create 5′ staggered ends, which potentially can facilitate precise gene replacement using non-homologous end joining (NHEJ), moreover it cleaves DNA at sites distal to the PAM. Such distal cleavage allows previously mutated sequences to be severed repeatedly, promoting homology-dependent repair (HDR) (Safari, F., Zare, K., Negandaripour, M., Barekati-Mowahed, M., and Ghasemi, Y. (2019) CRISPR Cpf1 proteins: structure, function and implications for genome editing. Cell Biosci., 9, 36.). To date three homologues of Cpfl, including Francisella novicida (FnCpf1), Acidaminococcus sp (AsCpf1), and Lachnospiraceae bacterium (LbCpf1) have been applied to genome engineering of different organisms.

### EXAMPLES

### General Strategy for Identifying and Generating Disease Citrus Plants via Gene Editing:

Stage 1: (i) Select candidate genes likely involved in disease susceptibility; (ii) obtain their DNA sequences by retrieving from genome database or sequencing; (iii) identify relatively conserved and critical regions in the targeted genes; (iv) design proper gRNAs to target the genes; (v) assemble the gRNA(s) with other essential components for effective gene editing (i.e. Cas9 and promoter, etc.) and efficient identification of Citrus cells, tissues and organs that carry these components (i.e. nptll gene and gfp gene); (vi) deliver the gene construct into Citrus cells via Agrobacterium tumefaciens mediated transformation or other approaches, or delivery the Cas9 protein and gRNA complex into Citrus cells; (vii) identify transgenic tissues shoots; (viii) micrograft shoot tips onto rootstock; (ix) obtain complete plantlets; (x) grow them in test tubes and then in the containers; (xi) isolate genomic DNA; (xi) amplify the targeted gene regions with specific primers; (xii) deep sequencing of the amplicons; (xiii) identify mutations and their frequencies; (xiv) inoculate mutants with Xanthomonas citri ssp. citri (Xcc) to determine their resistance to citrus canker; and (xv) identify mutants with little citrus canker disease symptoms and low Xcc bacterial cells.

Upon completing Stage 1 strategy, the following approach (Stage 2) is then conducted: Stage 2: (i) propagate citrus canker-resistant mutants; (ii) inoculate these mutants with Candidatus liberibacter asiaticus (CLas, the associated pathogen of huanglongbing or citrus greening); (iii) measure plant growth and CLas titers; and (iv) identify Citrus mutants resistant to both citrus canker and huanglongbing.

### Example 1

### Designing of Guide RNAs (gRNAs) to Target Specific Genes in Citrus

1. Identifying candidate genes for gene editing
A thorough literature review identified two genes, SWEET1 and DMR6, as candidate genes for gene editing.
SWEET=Sugars will eventually be exported transporters.
DMR=downy mildew resistance.

SWEET genes encode one of the several types of sugar transporters, and their proteins are located in the plasma membrane of cells, thus facilitating the efflux of sugars for a variety of purposes. These sugar transporters are often hijacked by plant pathogens for supply of sugars as a source of nutrients for the pathogens to infect plant cells and multiply within infected cells (Gupta, 2020). SWEET genes are now known in more than 30 plant species. In at least 10 of these species, some of the SWEET genes function as disease susceptibility genes (S genes) (Streubel et al., 2013). In rice, five naturally mutated SWEET genes confer resistance to Xanthomonas oryzae pv. oryzae (Xoo), the pathogen causing bacterial blight (Streubel et al., 2013). SWEET genes can also be used by some fungal pathogens, such as Botrytis cinera that cause gray mold disease in many plant species (Chen et al., 2010). Disrupting SWEET genes in rice has resulted in broad-spectrum resistance to bacterial blight caused by Xoo (Xu et al., 2019).

DMR6 gene encodes a salicylate 3-hydroxylase (Zhang et al., 2017). The DMR6 protein is a repressor of plant immunity (Zeilmaker et al., 2015). It regulates negatively the expression of plant defense genes such as PR-1, PR-2, and PR-5. It is required for susceptibility to a number of pathogens, including susceptibility to the downy mildew pathogen Hyaloperonospora in Arabidopsis, susceptibility to Pseudomonas syringae pv. tomato DC3000 (Zhang et al., 2017), and susceptibility to the oomycete Phytophthora capsici (Van Damme et al., 2005). CRISPR-Cas9 mediated mutagenesis of a DMR6 ortholog in tomato resulted in broad-spectrum resistance to P. syringae, P. capsica, and Xanthomonas gardneri and X. perforans (del Toledo Thomazella et al., 2016).

2. Coding sequences of SWEET1 and DMR6 from C. sinensis V1.1 (sweet orange genome) (https://phytozome.jgi.doe.gov/pz/portal.html#·info?alias=Org_Csinensis

The SWEET1 and DMR6 gene sequences were retrieved from Phytozome C. sinensis V1.1.

EXON 1, 3, 5 (bold)
EXON 2, 4, 6 (italics)
5′ UTR (underline)
>orange1.1g025686m_SWEET1
[coding sequence of six exons; 750 base]
(SEQ ID NO: 1)
ATGGATATTGCACATTTCTTGTTCGGTGTTTTTGGAAATGCTACTGCTTT
ATTTCTCTTCTTGGCTCCAACAATCACATTTAGGAGGATTGTAAGAAGGA
AATCCACAGAGCAATTCTCTGGAATTCCTTATGTGATGACCTTGCTTAAT
TGTCTCCTCTCTGCCTGGTATGGTCTCCCTTTTGTGTCGAAGAACAACAT
TTTGGTATCAACAATCAATGGAACTGGTTCAGCAATTGAGATCATATATG
TTTTGATCTTCCTACTCTTTGCGCCAAAGAAGGAGAAAGCGAAAATCTTT
GGGCTTTTCATGCTTGTTCTCACAGTCTTCGCTGCTGTTGCCTTGGTGTC
TCTCTTAGCCTTTCATGGCAACGCTAGAAAGATTTTCTGTGGCTTTGCTG
CTACCATTTTCTCTATTATCATGTATGCCTCACCTCTTTCTATCATGAGA
ATGGTGATTAAAACAAAGAGTGTTGAGTTCATGCCATTTTTCTTGTCACT
ATTTGTCTTCCTGTGTGGCACCTCTTGGTTCGTGTTTGGCCTTCTTGGCC
GTGACCCTTTTGTGGCCGTTCCCAATGGATTTGGGTGTGGGCTAGGCACA
ATGCAGTTGATTTTGTACTTCATCTATCACAAGAAGGGTGAGCCTGAGAA
ACCATCGGCAGCAAATGGGTCTGTTGAAATGGGCCAAGAGAAACCATTAG
AAGGAACCAAGATGGCTAACGGCAACGGGGCTCTAGTTGAACAAGTTTGA
>orange1.1g019665m_DMR6 [partial 5′ UTR, and the
coding sequence of four exons (1014 bases)]
(SEQ ID NO: 2)
GTGCGCTCATCTTCCTAGAAGGTGCAAATACTTTTGCTCTCTTAAATTTT
TTATCACTATGCTATCTGCATATATAGATAATTAATAAAATCAAATTATG
GATACCAAAGTTCTGTCCTCGGGAATCCGGTACACAAACTTGCCGGAGGG
CTACGTAAGGCCAGAATCGGAGAGACCAAATCTGTCTGAAGTTTCTGAAT
GCAAAAACGTTCCTGTTATTGATTTGGCTTGTGATGATAGAAGCTTAATT
GTGCAACAAGTTGCTGATGCTTGCAAGAATTATGGATTTTTCCAGGCGAT
AAATCACGAAGTGCCTTTAGAAACAGTGGAAAGAGTCTTAGAAGTGGCGA
AAGAGTTTTTTAACCTGCCAGTCGAGGAGAAGCTGAAGCTCTACTCAGAT
GATCCATCAAAGACAATGAGATTGTCAACGAGTTTTAATGTCAACAAGGA
GAAAGTTCACAATTGGAGAGATTACCTCAGACTTCACTGCTATCCTCTTG
ACAAGTATGTGCCTGAGTGGCCTTCAAATCCATCTACCTTCAAGGAATTT
GTAAGTACTTATTGTTCAGAAGTTCGAGGACTCGGCTATAGAGTGCTGGA
ATTAATATCAGAGAGCTTAGGTTTGGAAAAAGATTACATAAAGAAAGTGT
TGGGTGAACAAGGACAGCATATGGCTGTGAACTTTTACCCACCATGTCCA
GAGCCAGAGCTTACTTATGGATTGCCAGGACACACTGACCCAAATGCCCT
TACCATTTTATTGCAAGATTTAGAAGTGGCAGGTCTTCAAGTTCTCAAAG
ACGACAAATGGGTTGCTGTTAATCCCCTTCCTAATGCCTTTGTCATTAAT
ATTGGTGATCAACTGCAGGCATTGAGTAATGGAAGGTATAAGAGTGTGTG
GCATAGAGCTATTGTGAATGCTGAAAAGGCAAGGATGTCGGTTGCTTCAT
TCCTTTGCCCAAATAATGATGCAATGATCAGCCCTCCAAAAGCATTAACA
GAGGATGGATCTGGAGCCGTATACAGAGATTTCACATATGCTGAATATTA
CAGCAAGTTTTGGAGCAGAAACTTGGACCAAGAACATTGTTTGGAACTTT
TCAAGAACTAG

3. Designing oligonucleotide primers to amplify the corresponding genes from ‘Duncan’ grapefruit

Primers were designed to amplify at least one to two exons of each gene so that the more conserved regions in SWEET1 and DMR6 can be identified and used to design guide RNA (gRNA). gRNAs designed from the more conserved regions can be used to edit corresponding genes in a wider range of Citrus varieties using the CRISPR/Cas9 genome editing technology. The primers designed and used are listed Table 1.

EXON 1, 3, 5 (bold)
EXON 2, 4, 6 (italics)
5′ UTR
5′ Primer (forward) (underline)
3′ Primer (reverse) (italics underline)
>orange1.1g025686m_SWEET1
(SEQ ID NO: 1)
ATGGATATTGCACATTTCTTGTTCGGTGTTTTTGGAAATGCTACTGCTTT
ATTTCTCTTCTTGGCTCCAACAATCACATTTAGGAGGATTGTAAGAAGGA
AATCCACAGAGCAATTCTCTGGAATTCCTTATGTGATGACCTTGCTTAAT
TGTCTCCTCTCTGCCTGGTATGGTCTCCCTTTTGTGTCGAAGAACAACAT
TTTGGTATCAACAATCAATGGAACTGGTTCAGCAATTGAGATCATATATG
TTTTGATCTTCCTACTCTTTGCGCCAAAGAAGGAGAAAGCGAAAATCTTT
GGGCTTTTCATGCTTGTTCTCACAGTCTTCGCTGCTGTTGCCTTGGTGTC
TCTCTTAGCCTTTCATGGCAACGCTAGAAAGATTTTCTGTGGCTTTGCTG
CTACCATTTTCTCTATTATCATGTATGCCTCACCTCTTTCTATCATGAGA
ATGGTGATTAAAACAAAGAGTGTTGAGTTCATGCCATTTTTCTTGTCACT
ATTTGTCTTCCTGTGTGGCACCTCTTGGTTCGTGTTTGGCCTTCTTGGCC
GTGACCCTTTTGTGGCCGTTCCCAATGGATTTGGGTGTGGGCTAGGCACA
ATGCAGTTGATTTTGTACTTCATCTATCACAAGAAGGGTGAGCCTGAGAA
ACCATCGGCAGCAAATGGGTCTGTTGAAATGGGCCAAGAGAAACCATTAG
AAGGAACCAAGATGGCTAACGGCAACGGGGCTCTAGTTGAACAAGTTTGA
>orange1.1g019665m_DMR6
(SEQ ID NO: 2)
GTGCGCTCATCTTCCTAGAAGGTGCAAATACTTTTGCTCTCTTAAATTTT
TTATCACTATGCTATCTGCATATATAGATAATTAATAAAATCAAATTATG
GATACCAAAGTTCTGTCCTCGGGAATCCGGTACACAAACTGCCGGAGGGC
TACGTAAGGCCAGAATCGGAGAGACCAAATCTGTCTGAAGTTTCTGAATG
CAAAAACGTTCCTGTTATTGATTTGGCTTGTGATGATAGAAGCTTAATTG
TGCAACAAGTTGCTGATGCTTGCAAGAATTATGGATTTTTCCAGGCGATA
AATCACGAAGTGCCTTTAGAAACAGTGGAAAGAGTCTTAGAAGTGGCGAA
AGAGTTTTTTAACCTGCCAGTCGAGGAGAAGCTGAAGCTCTACTCAGATG
ATCCATCAAAGACAATGAGATTGTCAACGAGTTTTAATGTCAACAAGGAG
AAAGTTCACAATTGGAGAGATTACCTCAGACTTCACTGCTATCCTCTTGA
CAAGTATGTGCCTGAGTGGCCTTCAAATCCATCTACCTTCAAGGAATTTG
TAAGTACTTATTGTTCAGAAGTTCGAGGACTCGGCTATAGAGTGCTGGAA
TTAATATCAGAGAGCTTAGGTTTGGAAAAAGATTACATAAAGAAAGTGTT
GGGTGAACAAGGACAGCATATGGCTGTGAACTTTTACCCACCATGTCCAG
AGCCAGAGCTTACTTATGGATTGCCAGGACACACTGACCCAAATGCCCTT
ACCATTTTATTGCAAGATTTAGAAGTGGCAGGTCTTCAAGTTCTCAAAGA
CGACAAATGGGTTGCTGTTAATCCCCTTCCTAATGCCTTTGTCATTAATA
TTGGTGATCAACTGCAGGCATTGAGTAATGGAAGGTATAAGAGTGTGTGG
CATAGAGCTATTGTGAATGCTGAAAAGGCAAGGATGTCGGTTGCTTCATT
CCTTTGCCCAAATAATGATGCAATGATCAGCCCTCCAAAAGCATTAACAG
AGGATGGATCTGGAGCCGTATACAGAGATTTCACATATGCTGAATATTAC
AGCAAGTTTTGGAGCAGAAACTTGGACCAAGAACATTGTTTGGAACTTTT
CAAGAACTAG

TABLE 1
Primers designed for amplifying partial
gene sequences of SWEET1 and DMR6 in
‘Duncan’ grapefruit

| | | | Expected | |
|---|---|---|---|---|
| | | | product | SEQ |
| Primer | | Targeted | size | ID |
| name | Sequence | Gene | (bp) | NO |
| SWEET1_F1 | AGCGAAAATCT | SWEET1 | 153 bp | 3 |
| | TTGGGCTTT | | | |
| SWEET1_R1 | GAAAGAGGTGA | | | 4 |
| | GGCATACATGA | | | |
| DMR6_5p_F1 | TCATCTTCCTA | DMR6 | 469 bp | 5 |
| | GAAGGTGCAAA | | | |
| DMR6_R1 | CCTCGACTGGC | | | 6 |
| | AGGTTAAAA | | | |

4. Amplifying and obtaining partial sequences of the SWEET1 and DMR6 genes in ‘Duncan’ grapefruit (DGF).

5′ Primer (forward) site (underline)
3′ Primer (reverse) site (underline and italics)
>DGF_SWEET1_sequenced [153 bases, 100% identical
to Citrus sinensis SWEET1]
(SEQ ID NO: 7)
AGCGAAAATCTTTGGGCTTTTCATGCTTGTTCTCACAGTCTTCGCTGCTG
TTGCCTTGGTGTCTCTCTTAGCCTTTCATGGCAACGCTAGAAAGATTTTC
TGTGGCTTTGCTGCTACCATTTTCTCTATTATCATGTATGCCTCACCTCT
TTC
>DGF_DMR6_sequenced [469 bases, 97% nucleotide
identity to Citrus sinensis DMR6 gene]
(SEQ ID NO: 8)
TCATCTTCCTAGAAGGTGCAAATACTTTTGCGCTCTTTAATTTTTTATTT
ATCACTATGGTATCTGCATATATAGATAATTAATAAAATCAAATTATGGA
TACCAAAGTTCTGTCCTCGGGAATCCGGTACACAAACTTGCCGGAGGGCT
ACGTAAGGCCAGAATCGGAGAGACCAAATCTGTCTGAAGTTTCTGAATGC
GAAAACGTTCCTGTTATTGATTTGGCTTGTGATGATAGAAGCTTAATTGT
GCAACAAGTTGCTGATGCTTGCAAGAATTATGGATTTTTCCAGGTTTGTG
TTCAAAATTTCCTTGCCCTAAATCATTTGAGTTTGTTATATAGATGTATA
AACGCGTTAATCAGTCATATATTGGTTGCATATACAGGCGATAAATCACG
AAGTGCCTTTAGAAACAGTGGAAAGAGTCTTAGAAGTGGCGAAAGAGTTT
TTTAACCTGCCAGTCGAGG

5. Designing gRNAs to target the conserved regions of SWEET1 and DMR6 genes

The Benchling online software (www.benchling.com) was used to select the best gRNA(s) with high on-target scores and low off-target scores for editing the SWEET1 and DMR6 gene in Citrus.

One gRNA (SWT1_gRNA1; SEQ ID NO: 9) was designed for editing the SWEET1 gene in Citrus. This gRNA has 100% nucleotide identity over its entire length (23 nucleotides) with the SWEET1 gene in Citrus sinensis (sweet orange), Citrus clementina (Clementine mandarin), Citrus reticulata (mandarin and tangerine), Citrus ichangensis (Ichang papeda), Citrus aurantifolia, Poncirus trifoliata, and Atalantia buxifolia (relative of Citrus), whose genome sequences are available in the Citrus Genome Database (https://www.citrusgenomedb.org/). SWT1_gRNA1 has 100% nucleotide identity with the SWEET1 gene in Citrus maxima (pomelo) from its base 1 to 22, except its last base at the 3′ end. SWT1_gRNA1 also has 100% nucleotide identity with the SWEET1 gene in Citrus medica from its base 2 to 23, except its first base at the 5′ end. Because of this high sequence identity, SWT1_gRNA1 is able to guide Cas9 or other nucleases to the SWEET1 gene in many Citrus species and varieties, if not all Citrus species.

Two gRNAs (dmr6_gRNA1, SEQ ID NO: 10; dmr6_gRNA2, SEQ ID NO: 11) were designed for editing the DMR6 gene in Citrus. Both gRNAs have 100% nucleotide identity over its entire length (23 nucleotides) with the DMR6 gene in Citrus sinensis, Citrus clementina, Citrus reticulata, Citrus aurantifolia, Citrus maxima, Citrus ichangensis, and Poncirus trifoliata. Because of this high sequence identity, dmr6_gRNA1 and dmr6_gRNA2 are able to guide Cas9 or other nucleases to the DMR6 gene in essentially all Citrus species and varieties.

>DGF_SWEET1_sequenced
(SEQ ID NO: 7)
AGCGAAAATCTTTGGGCTTTTCATGCTTGTTCTCACAGTCTTCGCTGCTG
TTGCCT [symbol] CATGGCAACGCTAGAAAGATTTTC
TGTGGCTTTGCTGCTACCATTTTCTCTATTATCATGTATGCCTCACCTCT
TTC
>DGF_DMR6_sequenced
(SEQ ID NO: 8)
TCATCTTCCTAGAAGGTGCAAATACTTTTGCGCTCTTTAATTTTTTATTT
ATCACTATGGTATCTGCATATATAGATAATTAATAAAATCAAATTATGGA
TACCAAAGTTCTGTCCT [symbol] TTGCCGGAGGGCT
ACGTAAGGCCAGAATCGGAGAGACCAAATCTGTCTGAAGTTTCTGAATGC
GAAAACGTTCCTGTTATTGATTTGGCTTGTGATGATAGAAGCTTAATTGT
GCAACAAGTTGCTGATGCTTGCAAGAATTATGGATTTTTCCAGGTTTGTG
TTCAAAATTTCCTTGCCCTAAATCATTTGAGTTTGTTATATAGATGTATA
AACGCGTTAATCAGTCATATATTGGTTGCATATACAGGCGATAAATCACG
AAGTGCCTTTAGAAAC [symbol] TGGCGAAAGAGTTT
TTTAACCTGCCAGTCGAGG

TABLE 2
Sequence of guide RNAs (gRNAs) for targeting
SWEET1 and DMR6 genes in Citrus

| | | | gRNA | |
|---|---|---|---|---|
| | | | length | |
| | | | including | |
| | | | PAM | SEQ |
| | | | motif | ID |
| Gene | gRNA | Sequences | (bases) | NO |
| SWEET1 | SWT1_gRNA1 | CCTTGGTGTCTC | 23 | 9 |
| | | TCTTAGCCTTT | | |
| DMR6 | dmr6_gRNA1 | CCTCGGGAATCC | 23 | 10 |
| | | GGTACACAAAC | | |
| | dmr6_gRNA2 | AGTGGAAAGAGT | 23 | 11 |
| | | CTTAGAAGTGG | | |

Note to Table 2:
The protospacer adjacent motif (PAM) is indicated in blue letters.

### Example 2

### Developing Gene Constructs for Effective CRISPR/Cas9-Based Gene Editing

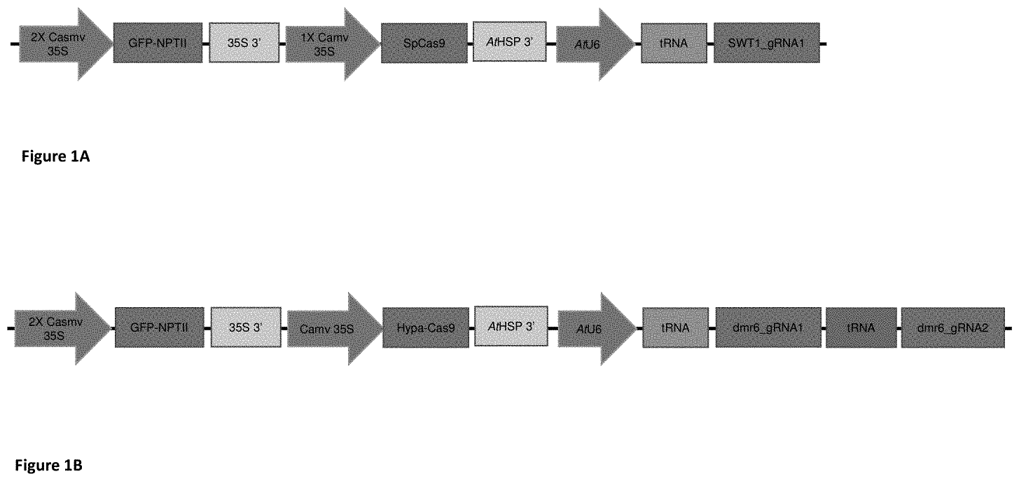

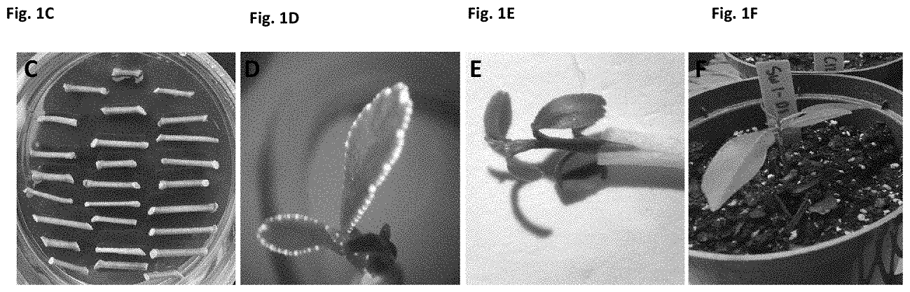

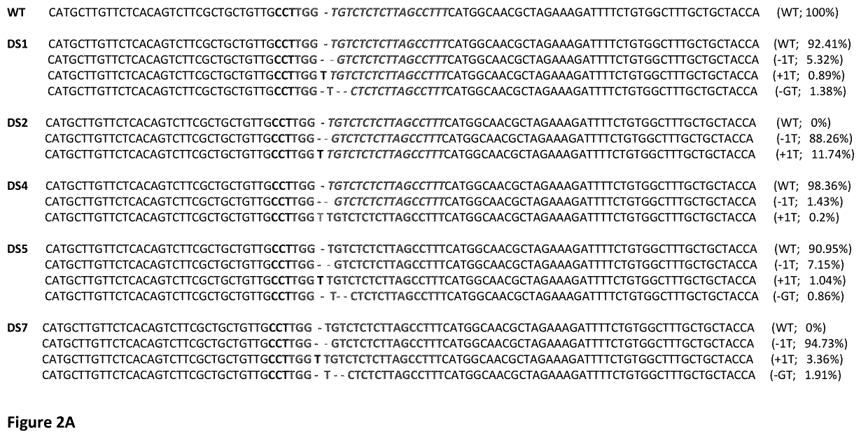

The plasmid vector pS1g1g2-Cas9 was assembled for editing the SWEET1 gene in Citrus, including ‘Duncan’ grapefruit. FIG. 1A shows a schematic diagram of this vector within the left and right transfer DNA (T-DNA) borders. In this gene editing vector, the GFP-NPTII fusion protein gene was driven under the 2X Casava mosaic virus (Casmv) 35S promoter, the SpCas9 gene was driven by the Cauliflower mosaic virus (Camv) 35S promoter, and the gRNA (SWT1_gRNA1) targeting the SWEET1 gene was driven under the Arabidopsis U6 promoter. To assemble the pS1g1g2-Cas9 vector, primers were designed for the gRNA (SWT1_gRNA1) with BsaI restriction sites to amplify ptRNA-scaff plasmid. The PCR product were gel-purified and ligated using the Golden Gate (GG) Assembly protocol with the BsaI enzyme. The GG product was amplified using S5Fok5-F and S5Fok3-R primers to add FokI restriction sites. The amplified PCR product was gel-purified and digested with FokI enzyme, and the pU6pro plasmid was digested with BsaI enzyme to insert the At U6 promoter into the gRNA construct. The FokI digested product and BsaI digested pU6pro plasmid were ligated with T4 DNA ligase to yield pU6-tRNA-gRNA plasmid. The ligated product was transformed to E. coli cells, and several E. coli colonies were selected. The plasmid DNA of these E. coli colony was sequenced to select the right clone with the U6 promoter and the gRNA construct (sequence verified clone). Finally, the Gateway assembly (LR reaction) was performed for the sequence verified clone and the pCas9-nptII-gfp plasmid to construct the final pS1g1g2-Cas9 vector. The pS1g1g2-Cas9 plasmid contained the green fluorescent protein (GFP) gene fused with the neomycin phosphotransferase (nptII) gene (as selectable marker), which is driven by the 2X Cassava mosaic virus (Casmv) 35S promoter and terminated with the 35S terminator. Expression of the SpCas9 gene was driven by the 35S promoter from Cauliflower mosaic virus (Camv) and terminated with the A. thaliana heat shock protein (AtHSP) terminator. The tRNA- gRNA-RNA-scaffold fragment was driven by the Arabidopsis thaliana ubiquitin 6 (AtU6) promoter.

The plasmid vector pD6g1g2-HyCas9 was assembled for editing DMR6 gene in Citrus, including ‘Duncan’ grapefruit and ‘Carrizo’ citrange. FIG. 1B shows a schematic diagram of this vector within the left and right transfer DNA (T-DNA) borders. In this vector, the GFP-NPTII fusion protein gene was driven under the 2X Casmv 35S promoter, the Hypa-Cas9 gene was driven under the Camv 35S promoter, and the two gRNAs (dmr6_gRNA1 and dmr6_gRNA2) targeting the DMR6 gene were driven under the Arabidopsis U6 promoter. To construct pD6g1g2-HyCas9 vector, the ptRNA-scaff plasmid with tRNA and RNA scaffold sequence was amplified with three pairs of primers: GG5-F&gRNAD1-R, gRNAD1-F&gRNAD2-R and gRNAD2-F&GG3-R. The three amplicons were assembled by using the Golden Gate (GG) Assembly protocol (Table S1). The GG assembled fragment was amplified with FG5-F&FG3-R primer pair to insert the FokI restriction sites, and the resulting fragment was digested with FokI enzyme. The pU6pro plasmid with the Arabidopsis U6 promoter was digested with BsaI enzyme and then ligated with FokI digested GG assembly product to yield the pU6-tRNA-gRNA plasmid. The ligated product was transformed into E. coli cells, and E. coli colonies were sequenced to select a clone with the U6 promoter and gRNA constructs. The pU6-tRNA-gRNA plasmid was used to perform gateway assembly (LR reaction) with the pHCas9-nptll-gfp plasmid. The gateway assembly product was transformed into DH5a E. coli cells to obtain the pD6g1g2-HyCas9 plasmid. The sequence of the plasmid was confirmed by whole plasmid sequencing. The pD6g1g2-HyCas9 plasmid had the green fluorescent protein (GFP) gene fused with the neomycin phosphotransferase (nptII) gene as selectable markers, which was driven under 2X Casmv 35S promoter and 35S terminator. The Hypa-Cas9 gene was driven by the Camv 35S promoter and A. thaliana heat shock protein (AtHSP) terminator. The tRNA-gRNA-RNA-scaffold fragment was driven by the A. thaliana Ubiquitin 6 (AtU6) promoter.

### Example 3:

### Delivering the Gene Constructs into Citrus Cells and Regenerating Citrus Shoots Containing Cas9 and gRNAs

The gene editing constructs or plasmids were delivered into Citrus cells through Agrobacterium tumefaciens-mediated stable transformation and regeneration of Citrus shoots carrying and expressing SpCas9 or Hypa-Cas9 gene and gRNAs was through organogenesis (FIG. 1C-1F). Details are as follows:

The above developed CRISPR/Cas9 plasmids, pS1 g1g2-Cas9 and pD6g1 g2-HyCas9, were electroporated separately into Agrobacterium tumefaciens strain EHA101 cells. Transformed Agrobacterium EHA101 was selected and used for delivering the gene editing plasmids into Citrus cells. In each experiment, the Agrobacterium cells were grown overnight at 28° C. Then the cells were harvested and adjusted to an optical density 0.5 (OD₆₀₀ 0.5) prior to use to deliver the gene editing components.

Seeds (peeled, containing apomictic nucellar embryos) of ‘Duncan’ grapefruit (DGF) and ‘Carrizo’ citrange (CRZ) were surface-sterilized with 0.5% sodium dichloroisocyanurate (NaDCC) and Tween-20 for 20 minutes and washed 3 times with autoclaved distilled and deionized water (ddH₂O). The seeds were sown on a solid culture medium in test tubes and germinated in dark, the seedlings were grown in dark for 4 weeks, and then the epicotyls of the seedlings were harvested and cut into 2-4 cm-long segments with slanting cut on both the ends.

The Citrus epicotyl segments (explants) were immersed in the Agrobacterium cell suspension for 2-5 minutes and then co-cultivated for 2 days on the co-cultivation media. After co-cultivation, the Agrobacterium-inoculated epicotyl segments were cultured on a selective regeneration medium containing kanamycin (100 mg/L) for 4-6 weeks until new green shoots emerged at the cut ends (FIG. 1C). The shoots were examined under a fluorescence microscope to identify green fluorescence-positive shoots (transformed or transgenic shoots).

To edit the SWEET1 gene in ‘Duncan’ grapefruit, a total of 1710 epicotyl segments were inoculated and co-cultivated with Agrobacterium harboring the pS1g1g2-Cas9 plasmid in multiple transformation experiments. These explants produced 11 GFP-positive shoots on the selective medium containing kanamycin (Table 3, FIG. 1D).

To edit the DMR6 in ‘Duncan’ grapefruit, a total of 1900 epicotyl segments were inoculated and co-cultivated with Agrobacterium harboring the pD6g1g2-HyCas9 plasmid in multiple transformation experiments. These explants produced nine GFP-positive shoots on the selective medium containing kanamycin (Table 3).

To edit the DMR6 in ‘Carrizo’ citrange, a total of 5320 epicotyl segments were inoculated and co-cultivated with Agrobacterium harboring the pD6g1g2-HyCas9 plasmid in multiple transformation experiments. These explants produced 57 GFP-positive shoots on the selective medium containing kanamycin (Table 3).

TABLE 3
Summary of Agrobacterium-mediated Transformation
of Citrus to Produce Mutants.

| | Gene | Explants | GFP positive | Transformation |
|---|---|---|---|---|
| Citrus | targeted | inoculated | shoots regenerated | efficiency (%) |
| ‘Duncan’ grapefruit | SWEET1 | 1710 | 11 | 0.64 |
| ‘Duncan’ grapefruit | DMR6 | 1900 | 9 | 0.47 |
| ‘Carrizo’ citrange | DMR6 | 5320 | 57 | 1.07 |

### Example 4

### Producing and Growing Complete Citrus Plants Expressing Cas9 and gRNAs

The GFP-positive shoots were micro-grafted on 3-week old, etiolated Varrizo' citrange rootstock seedlings (FIG. 1E). After 4 weeks, the grafted positive plants were transferred to soil in 4-inch containers and grown in a growth chamber until the shoot reached 10 to 15 cm in length. Then the citrus plants were transplanted into deep containers (30 cm deep) and grown in the greenhouse (FIG. 1F).

A total of six complete GFP-positive ‘Duncan’ grapefruit plants were successfully established in soil for the pS1g1g2-Cas9 construct targeting the SWEET1. Four complete GFP-positive ‘Duncan’ grapefruit plants were established in soil for the pD6g1g2-HyCas9 construct targeting the DMR6 gene in ‘Duncan’ grapefruit, while 16 complete GFP-positive Varrizo' citrange plants were established in soil for the same construct targeting the DMR6 gene in ‘Carrizo’ citrange.

TABLE 4
Summary of Production and Establishment of Transgenic Citrus Mutants.

| | | GFP positive | GFP-positive plants | GFP-positive plants |
|---|---|---|---|---|
| | Gene | shoots | produced by micro- | established in |
| Citrus | targeted | regenerated | grafting (no.) | containers (no.) |
| ‘Duncan’ grapefruit | SWEET1 | 11 | 7 | 6 |
| ‘Duncan’ grapefruit | DMR6 | 9 | 7 | 4 |
| ‘Carrizo’ citrange | DMR6 | 57 | 28 | 16 |

### Example 5

### Assessing Mutations and Mutation Frequencies at Targeted Genes

To reveal mutations and mutation frequencies in the GFP-positive lines, leaf tissue was collected from multiple leaves on each transgenic line and combined, genomic DNA was extracted, and the targeted SWEET1 or DMR6 gene regions were amplified by PCR with primers. Amplicons were recovered from agarose gels and subjected to next-generation deep sequencing at a commercial service laboratory. Sequencing output per line varied from 35,605 to 99,880 reads per sample. The sequencing reads were analyzed using the Geneious software to reveal the type of mutations and their frequencies. All sequence reads matched to the gRNA-targeted SWEET1 or DMR6 gene regions, except for the nucleotides downstream of the PAM site. Leaf tissue sampling, DNA isolation, PCR amplification, and DNA sequencing were repeated, once when plants were approximately 8 months old and again when they were about 36 months old. Non-transformed wildtype plants did not show any mutations at the targeted regions analyzed.

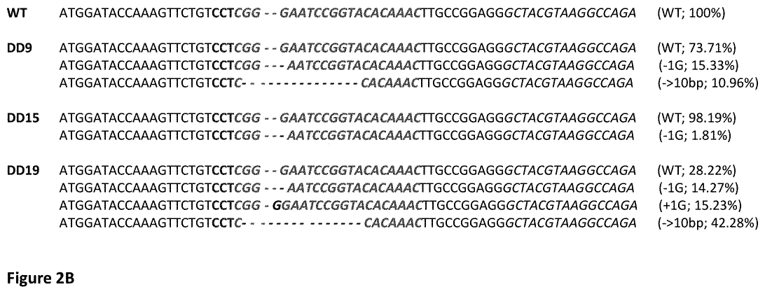

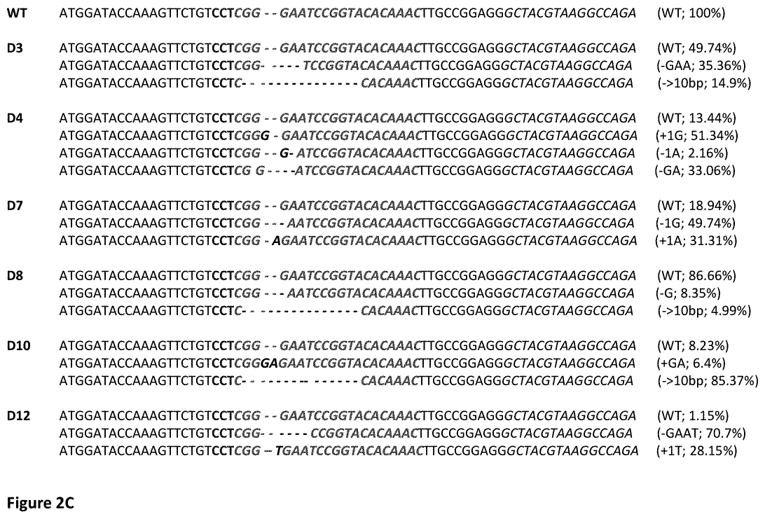

Five out of the six transgenic ‘Duncan’ grapefruit lines transformed with the pS1g1g2-Cas9 construct showed mutations in the SWEET1 gene. Overall, two lines (DS2 and DS7) showed a mutation rate of 100% while three lines had a mutation rate of less than 10%. Detailed results are presented in Table 5 and 7 and FIG. 2A.

Line DS1 (‘Duncan’ mutant): The SWEET1 gene in approximately 7.59% of ‘Duncan’ grapefruit cells are edited. The primary mutation is one-base deletion (of T) (5.32%). Minor portions of the cells contain one-base insertion mutation (of T) (0.89%) and two-base deletion (of GT) (1.38%).

Line DS2 (‘Duncan’ mutant): The SWEET1 gene in 100% of this line's cells are edited. The primary mutation in this line is one-base deletion (of T) in the SWEET1 gene (88.26%). A minor portion of the cells carry one-base insertion (of T) (11.74%).

Line DS4 (‘Duncan’ mutant): The SWEET1 gene in 1.63% of its cells are edited. The primary mutation in this line is one-base deletion (of T) (1.43%). A very minor portion of the cells contain one-base insertion (of T) (0.20%).

Line DS5 (‘Duncan’ mutant): The SWEET1 gene in 9.05% of its cells are edited. The primary mutation in this line is one-base deletion (of T) (7.15%). Minor portions of the cells contain one-base insertion (of T) (1.04%) or two-base deletion (of GT) (0.86%).

Line DS7 (‘Duncan’ mutant): The SWEET1 gene in 100% of its cells are edited. The primary mutation in this line is also one-base deletion (of T) (94.73%). Minor mutations include one-base insertion (of T) (3.36%) and two-base deletion (of GT) (1.91%).

TABLE 5
Type and Frequency of Mutations Induced in Five Different SWEET1-edited ‘Duncan’
Grapefruit (DGF) Lines, as revealed by deep sequencing.

| | | | Type and frequencies of mutations induced in citrus leaf cells of | | | | |
|---|---|---|---|---|---|---|---|
| | | Gene | mutant lines (%) | | | | |
| Lines | Citrus | targeted | −1 base | −2 bases | +1 base | Total mutation | “−T” mutation |
| DS1 | DGF | SWEET1 | 5.32 | 1.38 | 0.89 | 7.95 | 5.32 |
| DS2 | DGF | SWEET1 | 88.26 | | 11.74 | 100.00 | 88.26 |
| DS4 | DGF | SWEET1 | 1.43 | | 0.20 | 1.63 | 1.43 |
| DS5 | DGF | SWEET1 | 7.15 | 0.86 | 1.04 | 9.05 | 7.15 |
| DS7 | DGF | SWEET1 | 94.73 | 1.91 | 3.36 | 100.00 | 94.73 |

Note to Table 5:
“−1 base” = one-base deletion; “−2 bases” = two-base deletion; “+1 base” = one-base insertion; “−T” mutation = one-base deletion of T.

Three ‘Duncan’ grapefruit lines (DD9, DD15 and DD19) and six ‘Carrizo’ citrange lines (D3, D4, D7, D8, D10 and D12) are mutated in the DMR6 gene. Detailed results are presented in Table 6 and 7, and FIG. 2B and 2C.

Line DD9 (‘Duncan’ mutant): The DMR6 gene in this line are mutated in two regions, 26.29% of its cells in the dmr6-gRNA1-targeted region, and 12.17% of its cells in the dmr6-gRNA2-targeted region. Collectively, 26.29%, up to 38.36%, of its cells are mutated. This line of ‘Duncan’ grapefruit carries three types of mutation, one-base deletion (of G) (15.33%) and 10-base or larger deletion (10.96%) in the dmr6-gRNA1-targeted region, and one-base insertion (of G) in the dmr6-gRNA2-targeted region (12.17%).

Line DD15 (‘Duncan’ mutant): The DMR6 gene in 1.81% of its cells are mutated in the dmr6-gRNA1-targeted region. The only type of mutation is one-base deletion (G) (1.81%).

Line DD19 (‘Duncan mutant’): The DMR6 gene in 71.78% of its cells are mutated in the dmr6-gRNA1-targeted region. The primary mutation is ten-base or larger deletion (42.28%). Two types of minor mutations are one-base deletion (of G) (14.27%) and one-base insertion (G) (15.23%). A minor mutation is found in the dmr6-gRNA2-targeted region (2.39%).

Line D3 (‘Carrizo’ mutant): The DMR6 gene in 50.26% of its cells are mutated in the dmr6-gRNA1-targeted region and mutated in 1.12% of its cells in the dmr6-gRNA2-targeted region. Two types of mutations in the dmr6-gRNA1-targeted region are three-base deletion (of GAA) (35.36%) and ten-base or larger deletion (14.90%). In the dmr6-gRNA2-targeted region are multiple very minor mutations (total 1.21%).

Line D4 (‘Carrizo’ mutant): The DMR6 gene in 85.56% of its cells are mutated in the dmr6-gRNA1-targeted d region and mutated in 2.47% of its cells in the dmr6-gRNA2-targeted region. Two primary types of mutations in the dmr6-gRNA1-targeted region are one-base insertion (of G) (51.34%) and two-base deletion (of GA) (33.06%). Two minor mutations are one-base deletion (of A) (2.16%) in the dmr6-gRNA1-targeted region and five-base deletion (2.47%) in the dmr6-gRNA2-targeted region.

Line D7 (‘Carrizo’ mutant): The DMR6 gene in 81.05% of its cells are mutated in the dmr6-gRNA1-targeted region and mutated in 3.52% of its cells in the dmr6-gRNA2-targeted region. Two types of mutations in the dmr6-gRNA1-targeted region are one-base deletion (of G) (49.74%) and one-base insertion (of A) (31.31%). One minor mutation is five-base deletion (3.52%) in the dmr6-gRNA2-targeted region.

Line D8 (‘Carrizo’ mutant): The DMR6 gene in 13.34% of its cells are mutated in the dmr6-gRNA1-targeted region and mutated in 10.85% of its cells in the dmr6-gRNA2-targeted region. Two types of mutations in the dmr6-gRNA1-targeted region are one-base deletion (of G) (8.35%) and ten-base or larger deletion (4.99%). One type of mutation in the dmr6-gRNA2-targeted region is three-base insertion (10.85%).

Line D10 (‘Carrizo’ mutant): The DMR6 gene in 91.77% of its cells are mutated in the dmr6-gRNA1-targeted region. The primary type of mutation is ten-base or larger deletion (85.37%). One minor mutation is two-base insertion (of GA) (6.40%).

Line D12 (‘Carrizo’ mutant): The DMR6 gene in 98.85% of its cells are mutated in the dmr6-gRNA1-targeted region and mutated in 71.11% of its cells in the dmr6-gRNA2-targeted region. Two types of mutation in the dmr6-gRNA1-targeted region are four-base deletion (of GAAT) (70.70%) and one-base insertion (of T) (28.15%), and one type of mutation in the dmr6-gRNA2-targeted region is seven-base deletion (71.11%).

TABLE 6
Type and Frequency of Mutations Induced in Three Different DMR6-edited ‘Duncan’ Grapefruit
(DGF) Lines and Six Different DMR6-edited ‘Carrizo’ Citrange (CRZ), as revealed by deep sequencing.

| | | | Type and frequencies of mutations induced in citrus leaf cells of mutant lines (%) | | | | | | | | | | | |
|---|---|---|---|---|---|---|---|---|---|---|---|---|---|---|
| | | | dmr6-gRNA1-targeted region | | | | | | | | | | | |
| | | Gene | | | | | −10 or | | | dmr6-gRNA2-targeted region | | | | |
| Lines | Citrus | targeted | −1 | −2 | −3 | −4 | more | +1 | +2 | −5 | −7 | +1 | +3 | Mis. |
| DD9 | DGF | DMR6 | 15.33 | | | | 10.96 | | | | | 12.17 | | |
| DD15 | DGF | DMR6 | 1.81 | | | | | | | | | | | |
| DD19 | DGF | DMR6 | 14.27 | | | | 42.28 | 15.23 | | 2.39 | | | | |
| D3 | CRZ | DMR6 | | | 35.36 | | 14.90 | | | | | | | 1.21 |
| D4 | CRZ | DMR6 | 2.16 | 33.06 | | | | 51.34 | | 2.47 | | | | |
| D7 | CRZ | DMR6 | 49.74 | | | | | 31.31 | | 3.52 | | | | |
| D8 | CRZ | DMR6 | 8.35 | | | | 4.99 | | | | | | 10.85 | |
| D10 | CRZ | DMR6 | | | | | 85.37 | | 6.40 | | | | | |
| D12 | CRZ | DMR6 | | | | 70.70 | | 28.15 | | | 71.11 | | | |

Note to Table 6:
“−1”, “−2”, “−3”. “−4”, “−5”, or “−10” = one-, two-, three-, four-, five, or ten-base deletion; “+1”, “+2”, or “+3” = one-, two-, or three-base insertion; “Mis.” = Miscellaneous indels at low frequencies.

TABLE 7
Summary on Mutation and Frameshift Mutation Frequencies in SWEET1- and DMR6-edited Citrus
Lines (DGF = ‘Duncan’ Grapefruit, CRZ = ‘Carrizo’ Citrange), based on Table 5 and Table 6.

| | | | SWT-gRNA1 or | | | | |
|---|---|---|---|---|---|---|---|
| | | | dmr6-gRNA1 | | dmr6-gRNA2 | | Likely maximum |
| | | | | Frame shift | | Frame shift | total mutation |
| | | | Mutation | mutation | Mutation | mutation | frequency in |
| | | Gene | frequency | frequency | frequency | frequency | each mutant |
| Lines | Citrus | targeted | (%) | (%) | (%) | (%) | (%) |
| DS1 | DGF | SWEET1 | 7.59 | 100 | | | 7.59 |
| DS2 | DGF | SWEET1 | 100 | 100 | | | 100 |
| DS4 | DGF | SWEET1 | 1.63 | 100 | | | 1.63 |
| DS5 | DGF | SWEET1 | 9.05 | 100 | | | 9.05 |
| DS7 | DGF | SWEET1 | 100 | 100 | | | 100 |
| DD9 | DGF | DMR6 | 26.29 | 100 | 12.17 | 100 | 28.46 |
| DD15 | DGF | DMR6 | 1.81 | 100 | 0 | 0 | 1.81 |
| DD19 | DGF | DMR6 | 71.78 | 100 | 2.39 | 100 | 74.17 |
| D3 | CRZ | DMR6 | 50.26 | 14.90 | 1.21 | 100 | 51.47 |
| D4 | CRZ | DMR6 | 86.56 | 100 | 2.47 | 100 | 89.03 |
| D7 | CRZ | DMR6 | 81.06 | 100 | 3.52 | 100 | 84.58 |
| D8 | CRZ | DMR6 | 13.34 | 100 | 10.85 | 0 | 24.19 |
| D10 | CRZ | DMR6 | 91.77 | 100 | 0 | | 91.77 |
| D12 | CRZ | DMR6 | 98.85 | 100 | 71.11 | 100 | 100 |

### Example 6

### Assessing Resistance of Citrus Mutants to Citrus Canker Caused by Xanthomonas citri ssp. citri (Xcc)

‘Duncan’ grapefruit is highly susceptible to citrus canker caused by Xcc (Gottwald et al., 2002) and develops typical canker lesions 1-2 weeks after Xcc inoculation. ‘Carrizo’ citrange is also susceptible to citrus canker caused by Xcc (Gottwald et al., 2002). Generally, canker lesions start as pinpoint spots. Young lesions are raised or ‘pustular’, particularly on the lower leaf surface (Gottwald et al., 2002). Each lesion can reach a maximum size of 2 to 10 mm in diameter (Gottwald, 2000).

To assess the resistance of citrus mutants to citrus canker, X. citri ssp. citri (Xcc) strain 2004-00059, initially isolated by the Division of Plant Industry, Florida Department of Agriculture and Consumer Services (FDACS), was obtained from Dr. Jones Laboratory at the University of Florida (Gainesville, Fla., U.S.A.). The protocol for Xcc inoculum preparation, Xcc inoculation, and citrus canker lesion assessment were based on previously described methods (Jia et al., 2017b; Zhang et al., 2010) and modified as needed. Herein a brief description of the procedure is provided. Xcc inoculum was prepared by streaking stock culture onto nutrient agar plates and incubating the plates at 27° C. for 48 hours. The bacteria on the plates were collected, suspended in 0.1 M magnesium sulfate (MgSO₄) solution, and diluted to an OD₆₀₀ of 0.3. The diluted bacterial cell suspension [1×10⁸ colony-forming units (CFU)/mL] was used as inoculum for inoculating citrus mutant plants. Needleless 1-mL syringes were used to infiltrate 100 μL of the freshly prepared Xcc suspension into immature leaves at each site on either side of a leaf. The inoculated plants were maintained in a greenhouse and monitored daily for citrus canker lesion development. Each inoculated leaf was documented in photos regularly for up to 20 days after inoculation (DAI). During photo-taking, a ruler was placed by the side of the leaves to set the scale. Photos were analyzed in ImageJ software (https://imagej.nih.gove/ij) to calculate the lesion size (area) on each inoculated leaf. Xcc inoculation experiment was repeated three times when the mutant plants were approximately 12 to 14 months old. Similar results were obtained from the three inoculation experiments.

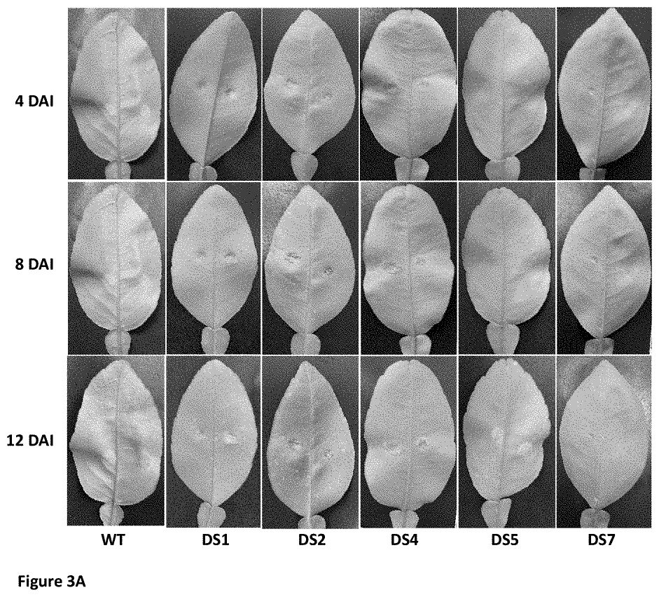

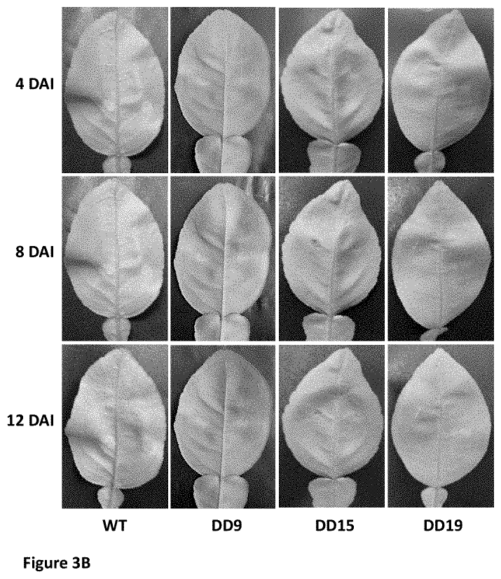

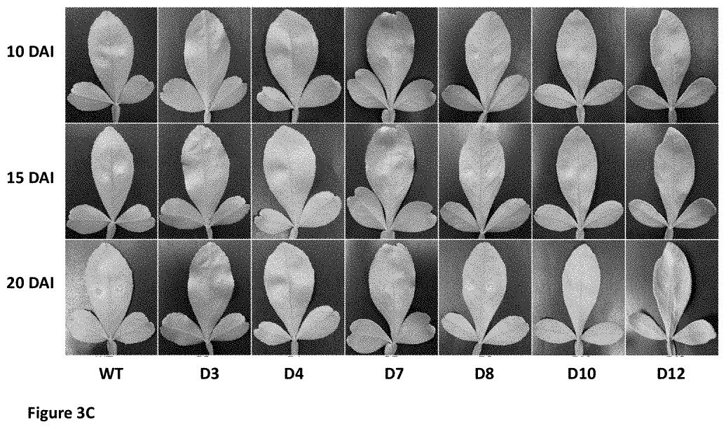

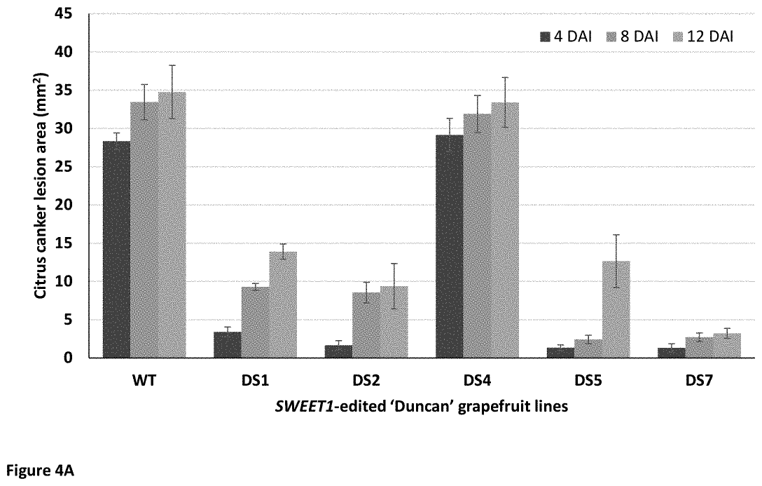

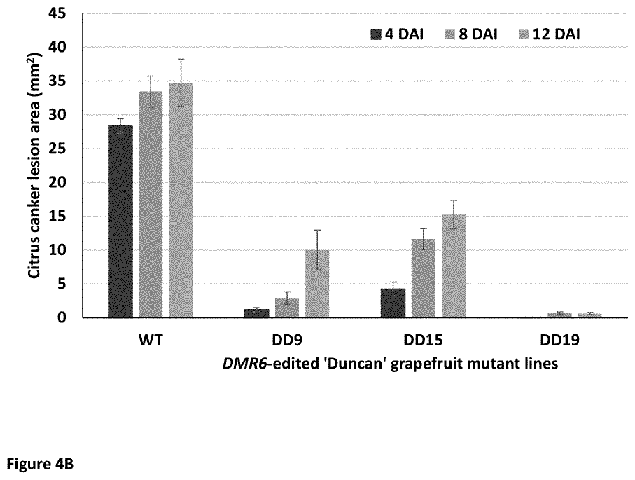

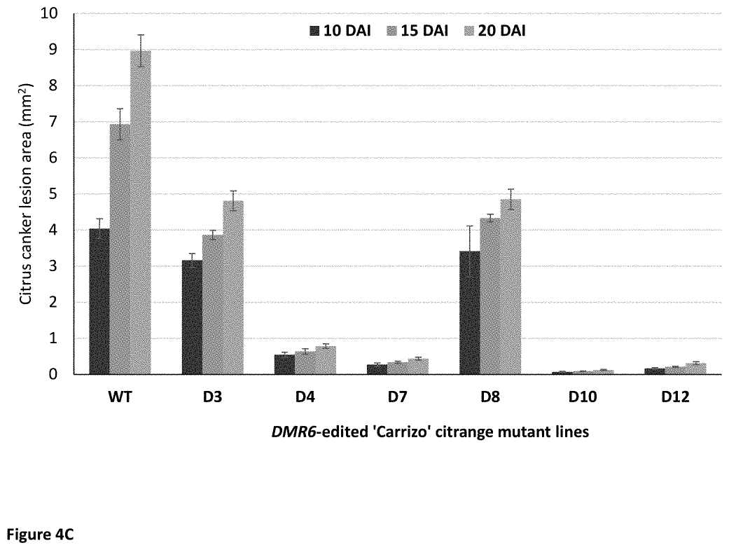

By 4 DAI, wildtype (non-transformed, non-mutant) ‘Duncan’ grapefruit showed large canker lesions on inoculated leaves at the infiltrated sites (Table 8, FIGS. 3A and 4A). After this, the lesions increased only gradually in size by 12 DAI. These results suggest that the leaves of wildtype ‘Duncan’ grapefruit were highly susceptible to Xcc and the concentration of Xcc bacterial cells applied was sufficient to induce rapid lesion development.

Compared to the wildtype ‘Duncan’ grapefruit, three out of the four independent SWEET1 mutant lines showed a dramatic reduction in canker lesion size (FIGS. 3A and 4A), or significantly increased resistance to Xcc.

Line DS2 and DS7: The average citrus canker lesion area in these mutant lines carrying fully mutated SWEET1 gene (100% cells mutated for SWEET1) was 94.1% and 95.3% smaller than the lesions on the wildtype plant at 4 DAI, 74.4% to 91.8% smaller at 8 DAI, and 73.0% to 90.7% smaller at 12 DAI (Table 8, FIG. 3A and 4A).

Lines DS1 and DS5: These lines carrying 7.59% or 9.05% SWEET1-mutated cells showed 94.1% and 95.3% reduction in lesion area at 4 DAI, 72.2% and 92.7% reduction at 8 DAI, and 60.0% and 63.6% reduction at 12 DAI (Table 8, FIG. 3A and 4A).

Line DS4: This mutant line DS4 carrying 1.63% mutation rate did not show any significant reduction in lesion area compared to the wildtype ‘Duncan’ grapefruit. This line developed large lesions by 4 DAI, about 15 fold larger than the lesions on leaves of mutant lines DS1, DS2, DS5 or DS7 (Table 8, FIG. 3A and 4A).

Line DD9 (‘Duncan’ grapefruit): Compared to the wildtype ‘Duncan’ grapefruit, this line carrying 26.29% to 28.46% mutation showed 95.7% lesion area reduction at 4 DAI, 91.3% lesion area reduction at 8 DAI, and 71.2% lesion area reduction at 12 DAI (Table 8, FIG. 3B and 4B).

Line DD15 (‘Duncan’ grapefruit): Compared to the wildtype ‘Duncan’ grapefruit, this line carrying 1.81% mutation showed 85.0% lesion area reduction at 4 DAI, 65.2% lesion area reduction at 8 DAI, and 56.1% lesion area reduction at 12 DAI (Table 8, FIG. 3B and 4B).

Line DD19 (‘Duncan’ grapefruit): Compared to the wildtype ‘Duncan’ grapefruit, this line carrying 71.78% to 74.17% mutation showed 99.8% lesion area reduction at 4 DAI, 97.9% lesion area reduction at 8 DAI, and 98.2% lesion area reduction at 12 DAI (Table 8, FIG. 3B and 4B).

Line D3 (‘Carrizo’ citrange): Compared to the wildtype ‘Carrizo’ citrange, this line carrying 50.26% to 51.47% mutation showed 21.8% lesion area reduction at 10 DAI, 44.2% lesion area reduction at 15 DAI, and 46.3% lesion area reduction at 20 DAI (Table 8, FIG. 3C and 4C).

Line D4 (‘Carrizo’ citrange): Compared to the wildtype ‘Carrizo’ citrange, this line carrying 86.56% to 89.03% mutation showed 86.5% lesion area reduction at 10 DAI, 90.8% lesion area reduction at 15 DAI, and 91.2% lesion area reduction at 20 DAI (Table 8, FIG. 3C and 4C).

Line D7 (‘Carrizo’ citrange): Compared to the wildtype ‘Carrizo’ citrange, this line carrying 81.06% to 84.58% mutation showed 93.2% lesion area reduction at 10 DAI, 95.1% lesion area reduction at 15 DAI, and 95.1% lesion area reduction at 20 DAI (Table 8, FIG. 3C and 4C).

Line D8 (‘Carrizo’ citrange): Compared to the wildtype ‘Carrizo’ citrange, this line carrying 13.34% to 24.19% mutation showed 15.5% lesion area reduction at 10 DAI, 37.5% lesion area reduction at 15 DAI, and 45.9% lesion area reduction at 20 DAI (Table 8, FIG. 3C and 4C).

Line D10 (‘Carrizo’ citrange): Compared to the wildtype ‘Carrizo’ citrange, this line carrying 91.77% mutation showed 98.2% lesion area reduction at 10 DAI, 98.7% lesion area reduction at 15 DAI, and 98.6% lesion area reduction at 20 DAI (Table 8, FIG. 3C and 4C).

Line D12 (‘Carrizo’ citrange): Compared to the wildtype ‘Carrizo’ citrange, this line carrying 98.85% to 100% mutation showed 95.9% lesion area reduction at 10 DAI, 96.9% lesion area reduction at 15 DAI, and 96.5% lesion area reduction at 20 DAI (Table 8, FIG. 3C and 4C).

TABLE 8
Lesion area (mm² from two inoculated sites per ‘Duncan’ grapefruit or ‘Carrizo’
citrange leaf) in wildtype (WT) and SWEET1-edited, or DMR6-edited ‘Duncan’ grapefruit
mutant lines 4, 8 and 12 days after inoculation (DAI) with Xanthomonas citri ssp.
citri (Xcc) and in wildtype and DMR6-edited ‘Carrizo’ citrange mutant lines
10, 15 and 20 days after inoculation with Xcc. Values shown are means from three experiments.

| | | | 4 DAI | | 8 DAI | | 12 DAI | |
|---|---|---|---|---|---|---|---|---|
| | | | Lesion | Reduction | Lesion | Reduction | Lesion | Reduction |
| | | | area | from | area | from | area | from |
| | | Gene | (mm² | wildtype | (mm² | wildtype | (mm² | wildtype |
| Lines | Citrus | edited | per leaf) | (%) | per leaf) | (%) | per leaf) | (%) |
| Wildtype | ‘Duncan’ | | 28.35 | | 33.45 | | 34.76 | |
| DS1 | ‘Duncan’ | SWEET1 | 3.41 | 88.0 | 9.30 | 72.2 | 13.91 | 60.0 |
| DS2 | ‘Duncan’ | SWEET1 | 1.67 | 94.1 | 8.56 | 74.4 | 9.39 | 73.0 |
| DS4 | ‘Duncan’ | SWEET1 | 29.14 | +2.8 | 31.91 | 4.6 | 33.41 | 3.9 |
| DS5 | ‘Duncan’ | SWEET1 | 1.35 | 95.3 | 2.44 | 92.7 | 12.66 | 63.6 |
| DS7 | ‘Duncan’ | SWEET1 | 1.33 | 95.3 | 2.73 | 91.8 | 3.24 | 90.7 |
| DD9 | ‘Duncan’ | DMR6 | 1.22 | 95.7 | 2.92 | 91.3 | 10.01 | 71.2 |
| DD15 | ‘Duncan’ | DMR6 | 4.25 | 85.0 | 11.65 | 65.2 | 15.25 | 56.1 |
| DD19 | ‘Duncan’ | DMR6 | 0.07 | 99.8 | 0.72 | 97.9 | 0.62 | 98.2 |

| | | |
|---|---|---|
| 10 DAI | 15 DAI | 20 DAI |

| | | | Lesion | Reduction | Lesion | Reduction | Lesion | Reduction |
|---|---|---|---|---|---|---|---|---|
| | | | area | from | area | from | area | from |
| | | Gene | (mm² | wildtype | (mm² | wildtype | (mm² | wildtype |
| Lines | Citrus | edited | per leaf) | (%) | per leaf) | (%) | per leaf) | (%) |
| Wildtype | ‘Carrizo’ | | 4.04 | | 6.93 | | 8.97 | |
| D3 | ‘Carrizo’ | DMR6 | 3.16 | 21.8 | 3.87 | 44.2 | 4.81 | 46.3 |
| D4 | ‘Carrizo’ | DMR6 | 0.55 | 86.5 | 0.64 | 90.8 | 0.79 | 91.2 |
| D7 | ‘Carrizo’ | DMR6 | 0.27 | 93.2 | 0.34 | 95.1 | 0.44 | 95.1 |
| D8 | ‘Carrizo’ | DMR6 | 3.41 | 15.5 | 4.33 | 37.5 | 4.85 | 45.9 |
| D10 | ‘Carrizo’ | DMR6 | 0.07 | 98.2 | 0.09 | 98.7 | 0.13 | 98.6 |
| D12 | ‘Carrizo’ | DMR6 | 0.17 | 95.9 | 0.21 | 96.9 | 0.31 | 96.5 |

### Example 7

### Assessing Bacterial Cell Populations in the Leaves of Citrus Mutants Inoculated with Xanthomonas citri ssp. citri (Xcc), the Pathogen of Citrus Canker

To confirm the mutant lines' resistance to Xcc, leaf tissues were collected from the Xcc-inoculated sites and the Xcc bacterial cells within the inoculated sites were counted by plating them on a selective medium in a series of dilution. Bacterial counting was conducted at 4, 8 and 12 days after Xcc inoculation for ‘Duncan’ grapefruit lines and at 10, 15 and 20 days after Xcc inoculation for ‘Carrizo’ citrange. Leaf discs were sampled from the inoculated sites, disinfected with 70% ethanol, and grounded in a Tissue Lyser II (Qiagen). The grounded leaf tissues were suspended in 1000 μL of autoclaved ddH₂O. Serial dilution of the sample up to 10⁸ was done by mixing 100 μL of the bacterial solution in 900 μL of autoclaved ddH₂O. The eight different serially diluted leaf tissue solutions were plated on nutrient agar plates, and bacterial colonies were counted after incubating the plated at 28° C. for 2 days. The experiment was repeated three times.

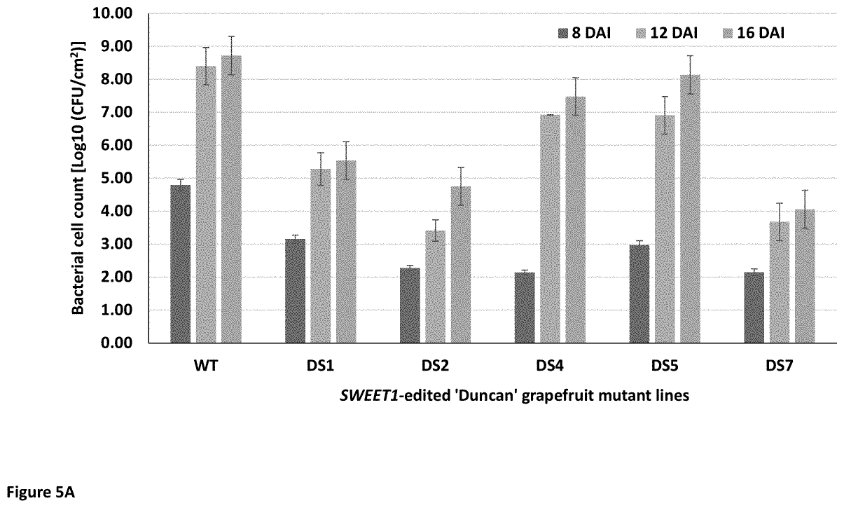

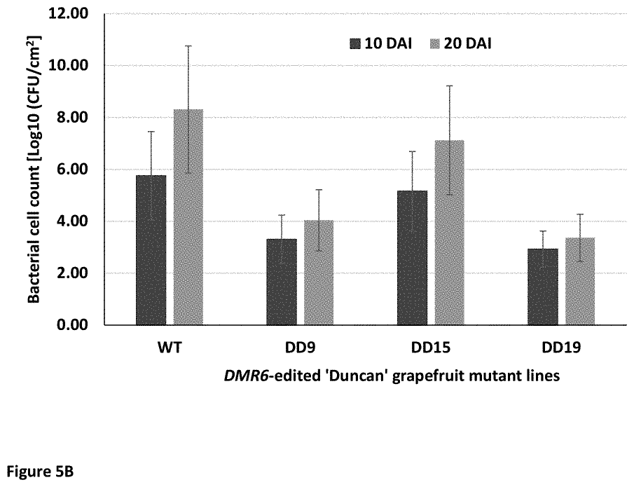

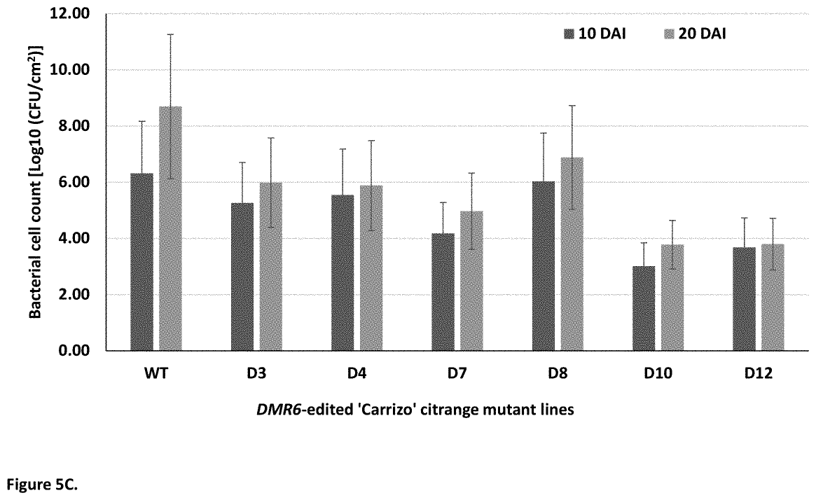

Xcc population exploded in the wildtype ‘Duncan’ leaves, between 8 DAI and 12 DAI, from a log10 CFU value (per cm²) of 4.73 to 7.82 (1222 fold increase; Table 9, FIG. 5A). From 12 DAI to 16 DAI, Xcc cell counts in the wildtype leaves remained high but increased only approximately 2 fold (FIG. 5A). The five mutant lines carrying mutations in the SWEET1 gene showed different Xcc bacterial cell counts and thus different levels of Xcc resistance (Table 9, FIG. 5A). The two fully mutated lines, DS2 and DS7, had the lowest Xcc cell counts at 8, 12, and 16 DAI. On inoculated leaves of DS2 and DS7, the log10 CFU per cm² was 2.13-2.26 at 8 DAI, 3.10-3.18 at 12 DAI, and 3.46-4.17 at 16 DAI. These Xcc cell counts represent 99.66% to 99.99% reduction, or 2.52 to 4.99 Log10 unit reduction, from the Xcc population in wildtype ‘Duncan’ grapefruit leaves (Table 9). The partially mutated lines DS1 and DS5, even the barely mutated line DS4, also showed dramatic reduction in Xcc cell counts compared to the wildtype (Table 9, FIG. 5A). DS1 and DS5 showed 74.1% % to 99.9% Xcc cell count reduction, or 0.59 to 3.18 Log10 unit reduction, at 8, 12 and 16 DAI. Interestingly, line DS4 (barely mutated) exhibited 94.3% to 99.8% Xcc population reduction, or 1.24 to 2.65 Log10 unit reduction, at 8, 12 and 16 DAI.

TABLE 9
Xanthomonas citri ssp. citri (Xcc) populations (bacterial cell counts) in leaves of wildtype
(WT) and SWEET1-edited ‘Duncan’ grapefruit 8, 12 and 16 days after inoculation (DAI) with Xcc.

| | | | | | | Xcc cell count reduction in | | |
|---|---|---|---|---|---|---|---|---|
| | | | Xcc cell counts (CFU/cm²) | | | mutants compared to WT (%) | | |
| | | Gene | [Log10 (CFU/cm²)] | | | (Log10 units) | | |
| Lines | Citrus | edited | 8 DAI | 12 DAI | 16 DAI | 8 DAI | 12 DAI | 16 DAI |
| WT | ‘Duncan’ | | 62,667 | 252,533,333 | 528,833,333 | | | |
| | | | (4.80) | (8.40) | (8.72) | | | |
| DS1 | ‘Duncan’ | SWEET1 | 1,433 | 191,567 | 346,500 | 97.71 | 99.92 | 99.93 |
| | | | (3.16) | (5.28) | (5.54) | (1.64) | (3.12) | 3.18) |
| DS2 | ‘Duncan’ | SWEET1 | 190 | 2,600 | 56,833 | 99.70 | >99.99 | 99.99 |
| | | | (2.28) | (3.41) | (4.75) | (2.52) | (4.99) | (3.97) |
| DS4 | ‘Duncan’ | SWEET1 | 140 | 8,366,667 | 30,120,000 | 99.78 | 96.69 | 94.30 |
| | | | (2.15) | (6.92) | (7.48) | (2.65) | (1.48) | (1.24) |
| DS5 | ‘Duncan’ | SWEET1 | 933 | 8,110,000 | 136,900,000 | 98.51 | 96.79 | 74.11 |
| | | | (2.97) | (6.91) | (8.14) | (1.83) | (1.49) | (0.59) |
| DS7 | ‘Duncan’ | SWEET1 | 142 | 4,763 | 11,330 | 99.77 | >99.99 | >99.99 |
| | | | (2.15) | (3.68) | (4.05) | (2.65) | (4.72) | (4.67) |

The three ‘Duncan’ grapefruit mutant lines carrying induced mutations in the DMR6 gene also showed different Xcc bacterial cell counts and thus different levels of Xcc resistance (Table 10, FIG. 5B). Mutated lines DD9 and DD19 had very low Xcc cell counts at 10 and 20 DAI. On inoculated leaves of DD9 and DD19, the log10 CFU per cm² was 3.30 or 2.93 at 10 DAI and 4.04 or 3.36 at 20 DAI (Table 10, FIG. 5B). These Xcc cell counts represent 99.65% to >99.99% reduction, or 2.45 to 4.95 Log10 unit reduction, from the Xcc population in wildtype ‘Duncan’ grapefruit leaves (Table 10). Mutated line DD15 showed 74.7% % to 93.6% Xcc cell count reduction, or 0.60 to 1.19 Log10 unit reduction, at 10 and 20 DAI (Table 10, FIG. 5B).

The six ‘Carrizo’ citrange mutant lines carrying mutations in the DMR6 gene showed different Xcc bacterial cell counts and thus different levels of Xcc resistance (Table 10, FIG. 5C). Three mutated lines, D7, D10 and D12, had the lowest Xcc cell counts at 10 and 20 DAI. On inoculated leaves of D7, D10 and D12, the log10 CFU per cm² was 3.01-4.18 at 10 DAI and 3.78-4.97 at 20 DAI. These Xcc cell counts represent 99.28% to >99.99% reduction, or 2.14 to 4.92 Log10 unit reduction, from the Xcc population in wildtype Carrizo citrange leaves (Table 10, FIG. 5C). Mutant lines D3 and D4 showed 83.17% to 99.85% Xcc cell count reduction, or 0.77 to 2.81 Log10 unit reduction, at 10 and 20 DAI (Table 10, FIG. 5C). Mutant line D8 carrying 13.34% DMR6-edited cells exhibited 48.51% and 98.45% Xcc population reduction, or 0.29 and 1.81 Log10 unit reduction, at 10 and 20 DAI, respectively (Table 10, FIG. 5C).

TABLE 10
Xanthomonas citri ssp. citri (Xcc) cell populations (bacterial cell
counts) in leaves of wildtype (WT) and DMR6-edited ‘Duncan’ grapefruit
and ‘Carrizo’ citrange 10 and 20 days after inoculation (DAI) with Xcc.

| | | | | | Xcc cell count reduction in | |
|---|---|---|---|---|---|---|
| | | | Xcc cell counts (CFU/cm²) | | mutants compared to WT (%) | |
| | | Gene | [Log10 (CFU/cm²)] | | (Log10 units) | |
| Lines | Citrus | edited | 10 DAI | 20 DAI | 10 DAI | 20 DAI |
| WT | ‘Duncan’ | | 569,000 | 204,666,667 | | |
| | | | (5.76) | (8.31) | | |
| DD9 | ‘Duncan’ | DMR6 | 2,000 | 10,933 | 99.65 | 99.99 |
| | | | (3.30) | (4.04) | (2.45) | (4.27) |
| DD15 | ‘Duncan’ | DMR6 | 143,733 | 13,200,000 | 74.74 | 93.55 |
| | | | (5.16) | (7.12) | (0.60) | (1.19) |
| DD19 | ‘Duncan’ | DMR6 | 850 | 2,300 | 99.85 | >99.99 |
| | | | (2.93) | (3.36) | (2.83) | (4.95) |
| WT | ‘Carrizo’ | | 2080000 | 495,000,000 | | |
| | | | (6.32) | (8.69) | | |
| D3 | ‘Carrizo’ | DMR6 | 183,333 | 966,667 | 91.19 | 99.80 |
| | | | (5.26) | (5.99) | (1.05) | (2.71) |
| D4 | ‘Carrizo’ | DMR6 | 350,000 | 766,667 | 83.17 | 99.85 |
| | | | (5.54) | (5.88) | (0.77) | (2.81) |
| D7 | ‘Carrizo’ | DMR6 | 15,000 | 93,333 | 99.28 | 99.98 |
| | | | (4.18) | (4.97) | (2.14) | (3.72) |
| D8 | ‘Carrizo’ | DMR6 | 1,071,000 | 7,666,667 | 48.51 | 98.45 |
| | | | (6.03) | (6.88) | (0.29) | (1.81) |
| D10 | ‘Carrizo’ | DMR6 | 1033 | 6,000 | 99.95 | >99.99 |
| | | | (3.01) | (3.78) | (3.30) | (4.92) |
| D12 | ‘Carrizo’ | DMR6 | 4,800 | 6,333 | 99.77 | >99.99 |
| | | | (3.68) | (3.80) | (2.64) | (4.89) |

### Example 8

### Determining Salicylic Acid (SA) Contents in Fresh Leaf Tissues in Citrus Mutants Before and After Inoculation with Xanthomonas citri ssp. citri

SA is as a key plant hormone for establishing resistance to many plant pathogens (An and Mou, 2011; Ding and Ding, 2020). Pathogen infection often leads to SA accumulation in infected leaves. SA accumulation often parallels to or precedes the increase in expression of plant defense genes and development of systemic acquired resistance (SAR) (An and Mou, 2011). Application of SA on citrus plants inhibited citrus canker, indicating that SA plays a role in the local defense against citrus canker bacteria (de Lima Silva et al., 2019).

Four mutant lines, DS2 and DS7 carrying the fully mutated SWEET1 gene, and DD9 and DD19 carrying the mutated DMR6 gene, and the wildtype ‘Duncan’ grapefruit were analyzed for SA content before and 24 hours after inoculation (24 HAI) with Xcc. Fresh leaves were sampled and grounded in liquid nitrogen. The powdered leaf tissues were shipped on dry ice to a commercial laboratory (Creative Proteomics, Shirley, N.Y.) and analyzed by liquid chromatography and mass spectrometry (LC-MS). For each mutant line or wildtype, two biological replicates were analyzed.

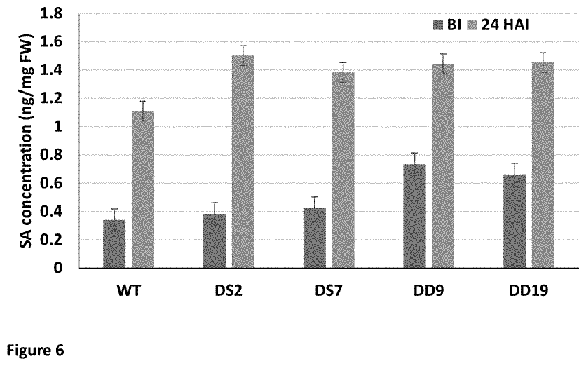

SA content in wildtype ‘Duncan’ grapefruit leaves was 0.34 and 1.11 ng/mg of fresh leaf immediately prior to Xcc inoculation and 24 HAI, respectively (Table 11 and FIG. 6). Thus, in response to Xcc inoculation, SA content in wildtype ‘Duncan’ grapefruit leaves increased rapidly and significantly (by 3.3 fold) within 24 hours.

Prior to Xcc inoculation, lines DS2 and DS7 contained 13.0% and 25.0% more SA in their leaves, compared to the wildtype. After Xcc inoculation, these lines also showed a rapid increase of SA content and contained 35.4% and 24.6% more SA than the non-edited wildtype line (Table 11 and FIG. 6).

Prior to Xcc inoculation, lines DD9 and DD19 contained 116.2% and 95.0% more SA in their leaves, compared to the wildtype. After Xcc inoculation, these lines also showed a rapid increase of SA content and contained 30.1% and 31.0% more SA than the non-edited wildtype line (Table 11 and FIG. 6).

TABLE 11
Salicylic acid (SA) contents in fresh leaf tissues of ‘Duncan’
grapefruit wildtype (WT) and four mutant lines before or 24 hours after
inoculation with Xanthomonas citri ssp. citri.

| | | | | | 24 hours after inoculation | |
|---|---|---|---|---|---|---|
| | | | Before inoculation | | with Xcc | |
| | | | SA content (ng/ | Change | SA content (ng/ | Change |
| | | | mg fresh | compared | mg fresh | compared |
| Lines | Citrus | Gene edited | weight) | to WT (%) | weight) | to WT (%) |
| WT | ‘Duncan’ | Non-edited | 0.340 | | 1.110 | |
| DS2 | ‘Duncan’ | SWEET1 | 0.384 | +13.0 | 1.502 | +35.4 |
| DS7 | ‘Duncan’ | SWEET1 | 0.425 | +25.0 | 1.383 | +24.7 |
| DD9 | ‘Duncan’ | DMR6 | 0.734 | +116.2 | 1.444 | +30.1 |
| DD19 | ‘Duncan’ | DMR6 | 0.662 | +95.0 | 1.453 | +31.0 |

### Example 9

### Determining the Relative Expression Level of the NPR1 in Fresh Leaf Tissues of Citrus Mutants Before and After Inoculation with Xanthomonas citri ssp. citri

The non-expressor of pathogenesis-related genes 1 (NPR1) plays a fundamental role in plant response to pathogen challenge (Backer et al., 2019). NPR1 acts as the master key to the plant defense signaling network, mediating cross-talk between the salicylic acid and jasmonic acid/ethylene response. Higher expression levels of NPR1 are essential for establishing the systemic acquired resistance (SAR) as well as induced systemic resistance (ISR). DMR6 gene is known to encode a SA hydrogenase.

Two DMR6-edited ‘Carrizo’ citrange lines and their wildtype ‘Carrizo’ plants were analyzed for the expression of NPR1 gene. The immature citrus leaves were inoculated with 10⁸ CFU/mL Xcc cell suspension, and discs of leaf tissues were sampled before inoculation, 10, 15 and 20 DAI for analysis of NPR1 gene expression. RNA was extracted using the RNeasy Mini Plus RNA extraction kit (Qiagen, Md., USA) and 1μg of RNA was converted into cDNA using the Superscript III First-strand cDNA synthesis kit (Invitrogen, Calif., USA). The quantitative real time RT-PCR was performed on Quantstudio 5 with PowerUP SYBR green master mix (Thermo Fisher Scientific, Waltham, Mass., USA) under the following conditions: 95° C. for 5 min for denaturation, 40 cycles at 95° C. for 15 s and 60° C. for 30 s and melt curve analysis at 95° C. for 15 s and 65-95° C. with 0.5° C. increment every 5 s. The relative expression of NPR1 gene was calculated using 2^{{Ct (GAPDH)-Ct (transgene)}}. Glyceraldehyde 3-phosphate dehydrogenase (GAPDH) gene was used as the internal reference. The gene expression analysis was performed with three biological replicates and two technical replicates.

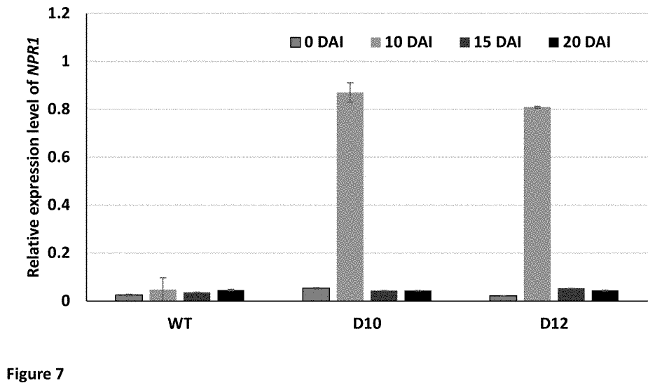

Before inoculation with Xcc cells, the expression levels of NPR1 gene in the wildtype ‘Carrizo’ citrange and two mutant lines (D10 and D12) were not significantly different, ranging from 0.021 to 0.053 (Table 12, FIG. 7). However, at 10 DAI, the NPR1 gene was expressed 16-17 fold higher in both mutant lines than in the wildtype (Table 12, FIG. 7). Subsequently, the expression level of NPR1 gene in the mutant lines dropped and became not significantly different from that in the wildtype at 20 DAI.

TABLE 12
Relative expression level of NPR1 gene in wildtype (WT) and DMR6-edited ‘Carrizo’ citrange
lines at 0, 10, 15 and 20 days after inoculation (DAI) with Xanthomonas citri ssp. citri.

| | | | 0 DAI | | 10 DAI | | 15 DAI | | 20 DAI | |
|---|---|---|---|---|---|---|---|---|---|---|
| | | | | Change | | Change | | Change | | Change |
| | | Gene | | over WT | | over WT | | over WT | | over WT |
| Lines | Citrus | edited | Mean | (%) | Mean | (%) | Mean | (%) | Mean | (%) |
| WT | ‘Carrizo’ | Non-edited | 0.024 | | 0.047 | | 0.036 | | 0.045 | |
| D10 | ‘Carrizo’ | DMR6 | 0.053 | +113.2 | 0.871 | +1736.9 | 0.043 | +20.2 | 0.044 | −4.8 |
| D12 | ‘Carrizo’ | DMR6 | 0.021 | −14.2 | 0.809 | +1606.5 | 0.053 | +45.2 | 0.044 | −3.6 |

### REFERENCES

Albrecht, U. and Bowman, K. D. 20.19. Reciprocal influences of rootstock and scion citrus cultivars challenged with Ca. Liberibacter asiaticus. Scientia Horticulturae 254:133-142.

Almeida, R. P. P. Nascirnento, F. E. Chau, J, Prado, S. S., Tsai, C. W., Lopes, S. A., and Lopes, J. R. S. 2008. Genetic structure and biology of Xylella filstidiosa strains causing disease in citrus and coffee in Brazil. Applied and Environmental Microbiology 3690-3701. doi:10.1128/AEM.02388-07

An, C., and Mou, Z. 2011. Salicylic acid and its function in plant immunity. Journal of Integrative Plant Biology 5:3:412-428.

Backer, R., Naidoo, S., and van den Berg, N. 2019. The nonexpressor of pathogenesis-related genes 1 (NPR1) and related family: mechanistic insights in plant disease resistance. Frontiers in Plant Science, 10, 102.

Chen, L. Q., Hou, B. H., Lalonde, S., Takanaga, H., Hartung, M. L., Qu, X. Q., Guo, W. J., Kim, J. G., Underwood, W., Chaudhuri, B. and Chermak, D., 2010. Sugar transporters for intercellular exchange and nutrition of pathogens. Nature 468(7323), pp.527-532.

Coletta-Filho, H. D., Castillo, A.I., Laranjeira, FF., de Andrade, E. C., Silva, N. T., de Souza, A. A., Bossi, M. E., Almeida, R. P. P., and Lopes, J. R. S. 2020. Citrus variegated chlorosis: An overview of 30 years of research and disease management. Tropical Plant Pathology 45:175-191.

de Toledo Thomazella, D. P., Brail, Q., Dahlbeck, D. and Staskawicz, B., 2016. CRISPR-Cas9 mediated mutagenesis of a DMR6 ortholog in tomato confers broad-spectrum disease resistance. BioRxiv, p.064824.

Ference, C. M., Gochez, A. M., Behlau, F., Wang, N., Graham, J. H., and Jones, J. B. 2018. Recent advances in the understanding of Xanthomonas citri ssp. citri pathogenesis and citrus canker disease management. Molecular Plant Pathology 19(6):1302-1318.

Gochez, A. M., Behlau, F., Singh, R., Ong, K., Whilby, L., and Jones, J. B. 2020. Panorama of citrus canker in the United States. Tropical Plant Pathology. https://doi.org/10.1007/s40858-020-00355-8

Gottwald, T. R., Graham, J. H., and. Schubert, T. S. 2002. Citrus canker: The pathogen and its impact. Plant Health Progress 3(1). https://doi.org/10.10904/OGO-2002-0812-01-RV

Graham, J. H., Gottwald, T. R., Cubero, J, and Achor, D. S. 2004. Xanthomonas axonopodis pv. citri: Factors affecting successful eradication of citrus canker. Molecular Plant Pathology 5(1):1 -15.

Gupta, P.K. 2020. SWEET genes for disease resistance in plants. Trends in Genetics. DOI: https://doi.org/10.1016/j.tig.2020.08.007

Ibanez, F., Suh, J. H., Wang, Y., Rivera, M., Setamou, M., & Stelinski, L. L. (2020). Salicylic acid mediated immune response of Citrus sinensis to varying frequencies of herbivory and pathogen inoculation. bioRxiv. doi: https://doi.org/10.1101/2020.08.04.235911

Jia, H., Orbovie, V., and Wang, N. (2019) CRISPR-LbCas12a-mediated modification of citrus. Plant Biotechnology Journal, pbi.13109.

Jia, H., Xu, J., Orbovie, V., Zhang, Y., and Wang, N. (2017a) Editing citrus genome via SaCas9/sgRNA system. Frontier in Plant Science, 8, 1-9.

Jia, H., Zhang, Y., Orbović, V., Xu, J., White, F. F., Jones, J. B., and Wang, N. (2017b) Genome editing of the disease susceptibility gene CsLOB1 in citrus confers resistance to citrus canker. Plant Biotechnology Journal, 15, 817-823.

Moore, G. A., Febres, V. J., Niblett, C. L., Luth, D., McCaffery, M., and Garnsey, S. M. 2000. Agrobacterium-mediated transformation of grapefruit (Citrus paradisi Macf.) with genes from citrus tristeza virus. Acta Horticulturae 535:237-244.

National Research Council. 2010. Strategic Planning for the Florida Citrus Industry: Addressing Citrus Greening Disease. Washington, D.C.: The National Academies Press.

Omar, A. A., Murata, M. M., El-Shamy, H. A., Graham, J. H., and Grosser, J.W. 2018. Enhanced resistance to citrus canker in transgenic mandarin expressing Xa21 from rice. Transgenic Research 27:179-191.

Peng, A., Chen, S., Lei, T., Xu, L., He, Y., Wu, L., et al. (2017) Engineering canker-resistant plants through CRISPR/Cas9-targeted editing of the susceptibility gene CsLOB1 promoter in citrus. Plant Biotechnology Journal, 1-11.

Sendin et al. (2017) Inducible expression of Bs2 R gene from Capsicum chacoense in sweet orange (Citrus sinensis L. Osbeck) confers enhanced resistance to citrus canker disease. Plant Molecular Biology 93:607-621.

Shi, Q., Febres, V. J., Jones, J. B., and Moore, G. A. 2016. A survey of FLS2 genes from multiple citrus species indicate candidates for enhancing disease resistance to Xanthomonas citri ssp. citri. Horticulture Research 3:16022. http://dx.doi.org/10.1038/hortres.2016.22

Soares, J. M., Tanwir, S. E., Grosser, J. W., and Dutt, M. 2020. Development of genetically modified citrus plants for the control of citrus canker and huanglongbing. Tropical Plant Pathology 45:237-250.

Streubel, J., Pesce, C., Hutin, M., Koebnik, R., Boch, J. and Szurek, B., 2013. Five phylogenetically close rice SWEET genes confer TAL effector-mediated susceptibility to Xanthomonas oryzae pv. oryzae. New Phytologist, 200(3), pp.808-819.

Wang, Y., Zhou, L, Yu, X., Stover, E Luo, & Duan, Y. (2016). Transcriptome profiling of Huanglongbing (HLB) tolerant and susceptible citrus plants reveals the role of basal resistance in FILM tolerance. Frontiers in Plant Science, 7, 933.

Wang, L., Chen, S., Peng, A., Xie, Z., He, Y., and Zou, X. 2019. CRISPR/Cas9-mediated editing of CsWRKY22 reduces susceptibility to Xanthomonas citri subsp. citri in Wanjincheng orange (Citrus sinensis (L.) Osbeck). Plant Biotechnology Reports. https://doi.org/10.1007/s11816-019-00556-x.

Xu, Z., Xu, X., Gong, Q., Li, Z., Li, Y., Wang, S., Yang, Y., Ma, W., Liu, L., Zhu, B. and Zou, L. 2019. Engineering broad-spectrum bacterial blight resistance by simultaneously disrupting variable TALE-binding elements of multiple susceptibility genes in rice. Molecular Plant, 12(11), pp.1434-1446.

Zeiltnaker, T., Ludwig, N. R., Elberse, J., Seidl, M. F., Berke, L., Van Doom, A., Schuurink, R. C., Snel, B. and Van den Ackerveken, G., 2015. DOWNY MILDEW RESISTANT 6 and DMR 6-LIKE OXYGENASE 1 are partially redundant but distinct suppressors of immunity in Arabidopsis. The Plant Journal, 81(2), pp.21.0-222,

Zhang, Y., Zhao, L., Zhao, J., Li, Y., Wang, J., Guo, R., Gan, S., Liu, C. J., & Zhang, K. (2017). S5H/DMR6 encodes a salicylic acid 5-hydroxylase that fine-tunes salicylic acid homeostasis, Plant Physiology, 175(3), 1082-1093. https://doi.org/10.1104/pp.17.00695

Zhang, X., Francis, M. I., Dawson, W. O., Graham, J. H., Orbović, V., Triplett, E. W., & Mou, Z. (2010). Over-expression of the Arabidopsis NPR1 gene in citrus increases resistance to citrus canker. European Journal of Plant Pathology, 128(1), 91-100.

1. A method of altering DNA sequence and gene structure of at least one gene product of a gene comprising
introducing into a citrus plant cell an engineered, non-naturally occurring gene editing system comprising one or more vectors, said citrus plant cell containing and expressing a DNA molecule encoding the gene and comprising a target sequence, said system comprising: (a) a first regulatory element operable in a plant cell operably linked to a nucleotide sequence encoding a Type-II CRISPR-associated nuclease, and (b) a second regulatory element operable in a plant cell operably linked to at least one nucleotide sequence encoding a CRISPR-Cas system guide RNA (gRNA) that hybridizes with the target sequence,
wherein components (a) and (b) are located on same or different vectors of the system,
wherein the CRISPR-associated nuclease cleaves the DNA molecule such that the DNA sequence, gene structure and/or expression of the gene is altered; and
wherein the gene is SWEET1 or DMR6.

2. The method of claim 1, wherein the coding sequence of the DNA molecule (SWEET1 gene) comprises SEQ ID NO: 1 or SEQ ID NO.9.

3. The method of claim 1, wherein the coding sequence of the DNA molecule comprises SEQ ID NO: 2, SEQ ID NO: 9 or SEQ ID NO: 10.

4. The method of claim 1 wherein said sequence encoding a Type-II CRISPR-associated nuclease are operably linked to a terminator sequence functional in a plant cell.

5. The method of any of claims 1, wherein said type II CRISPR-associated nuclease is Cas9.

6. The method of claim 1, wherein said second regulatory element comprises a DNA-dependent RNA polymerase III (Pol III) promoter sequence.

7. The method of claim 6, wherein said Pol III promoter sequence comprises an Arabidopsis U6 promoter nucleotide sequence.

8. The method of claim 7, wherein said Pol III promoter comprised of an Arabidopsis U6 promoter nucleotide sequence. .

9. The method of claim 1, wherein the gRNA is CCTTGGTGTCTCTCTTAGCCTTT.

10. The method of claim 1, wherein the gRNA is CCTCGGGAATCCGGTACACAAAC or AGTGGAAAGAGTCTTAGAAGTGG, or a combination.

11. A modified plant cell produced by the method of claim 1.

12. A plant comprising the plant cell of claim 11.

13. Seed, embryo, pollen, flower bud, flower, fruit, stem, bud, leaf, and/or root of the plant of claim 12.

14. A method of altering DNA sequence and gene structure and reducing expression of at least one gene product of a gene comprising introducing into a citrus plant cell a CRISPR-Cas-ribonucleoprotein complex (CRISPR-Cas-RNP), said citrus plant cell containing and expressing a DNA molecule encoding the gene and comprising a target sequence, wherein said CRISPR-Cas-RNP comprises a CRISPR-Cas system guide RNA (gRNA) that hybridizes with the target sequence, and a class-II CRISPR-associated nuclease, wherein the gene comprises SWEET1 or DMR6.

15. (canceled)

16. The method 14, wherein the class II CRISPR-associated nuclease comprises cfp1.

17. The method of claim 14, wherein the cfp1 is at least one selected from the group consisting of FnCpf1 from Francisella novicida, AsCpf1 from Acidaminococcus sp, and LbCpf1 from Lachnospiraceae bacterium.

18. (canceled)

19. A modified plant cell produced by the method of claim 14.

20. A plant comprising the plant cell of claim 19.

21. (canceled)

22.-32. (canceled)

33. A method of increasing resistance against citrus canker and Huanglongbing disease in a citrus plant comprising changing DNA sequence and gene structure and/or reducing expression of the SWEET1 gene or DMR6 gene, or both in cells of the citrus plant.

34. The method of claim 33, wherein reducing expression comprises imparting a mutation in the SWEET1 gene and/or DMR6 gene.

35. The method of claim 34, wherein imparting a mutation comprises introducing into one or more cells of the citrus plant a gene editing system comprising one or more vectors, comprising: (a) a first regulatory element operable in a plant cell operably linked to a nucleotide sequence encoding a Type-II CRISPR-associated nuclease, and (b) a second regulatory element operable in a plant cell operably linked to at least one nucleotide sequence encoding a CRISPR-Cas system guide RNA (gRNA) that hybridizes with a portion of the coding sequence, promoter and/or 5′ untranslated region of the SWEET1 gene and/or DMR6 gene.

36. The method of claim 34, wherein imparting a mutation comprises introducing into one or more cells of the citrus plant a CRISPR-Cas-ribonucleoprotein complex (CRISPR-Cas-RNP) comprising a CRISPR-Cas system guide RNA (gRNA) that hybridizes with a portion of the coding sequence, promoter and/or 5′ untranslated region of the SWEET1 gene and/or DMR6 gene, and a class-II CRISPR-associated nuclease.

37. (canceled)

38. (canceled)
